(19) United States
(12) Patent Application Publication
(10) Pub. No.: US 2026/0294243 A1
(43) Pub. Date: Oct. 1, 2026

(54) LIGHT MANAGEMENT FOR A VISION SCREENING DEVICE

(71) Applicant: Welch Allyn, Inc., Skaneateles Falls, NY (US)

(72) Inventors: Vivian L. Hunter, Auburn, NY (US); Joshua F. Hess, Moravia, NY (US); Scott A. Martin, Camillus, NY (US)

(21) Appl. No.: 19/629,989

(22) Filed: Mar. 26, 2026

Related U.S. Application Data
(60) Provisional application No. 63/779,848, filed on Mar. 28, 2025.

(51) Int. Cl. A61B 3/15; A61B 3/18

(52) U.S. Cl. CPC A61B 3/156; A61B 3/18

(57) ABSTRACT

A vision screening device may include a housing having an emission surface and enclose a first emitter with one or more light sources. The vision screening device may include a reflector connected to the first emitter and partially surrounding the one or more light sources, the reflector configured to reflect from the one or more light sources out of the emission surface. The vision screening device further includes a polarizer coupled to the reflector at an output side of the reflector and a beam splitter positioned between the polarizer and the emission surface such that light from the reflector passes through the beam splitter and reflected light received through the emission surface is directed at an angle relative to an incident angle of the reflected light.

### CROSS REFERENCE TO RELATED APPLICATIONS

This application claims priority to U.S. Provisional Application No. 63/779,848, titled “LIGHT MANAGEMENT FOR A VISION SCREENING DEVICE”, filed Mar. 28, 2025, which is hereby incorporated by reference in its entirety.

### TECHNICAL FIELD

This application is directed to medical equipment. In particular, this application is directed to a light management system for a vision screening device, and associated systems and methods, for controlling and conditioning light as part of operations for detection and assessment of diseases and disorders of the eye and performing visual acuity exams for a patient.

### BACKGROUND

Vision screening typically includes screening for diseases of the eye and visual acuity examinations. Such screening may include determining a visual acuity for each eye of a patient as well as screening using a plurality of both visible light, object recognition, color recognition, and infrared light tests to diagnosis a wide variety of potential eye diseases. For example, a transillumination test such as the Brückner red reflex test may be performed. During the red reflex test, the clinician illuminates the eye of the patient with visible light using an ophthalmoscope and examines the color and other characteristics of the light reflected back by the choroid and the retinal surfaces of the eye. Various diseases and abnormalities of the eyes can be detected using this test, such as corneal or media opacities, cataracts, and retinal abnormalities including tumors and retinoblastoma.

In addition, vision screening typically also includes one or more tests to determine various deficiencies associated with the patient's eyes. Such vision tests may include, for example, refractive error tests, accommodation tests, visual acuity tests, color vision screening and the like. One illustrative vision test involves the measurement of high contrast visual acuity. Visual acuity is a quantitative assessment of the ability to resolve high contrast optotypes. In the United States, the measurement is recorded in a ratio, such as 20/20, 20/40, 20/200, and so on. The ratio 20/20 indicates that at 20 feet, an individual is able to resolve a high contrast black letter which subtends 5 minutes of arc against a white background. From a test distance of 20 feet away, the 20/20 letter is 8.87 mm tall. The ratio 20/40 indicates that the individual can resolve a letter which is twice the size as the 20/20 benchmark. The ratio 20/200 means that the individual can resolve a letter that is ten times the size as the 20/20 benchmark.

Some of the vision screening tests require the use of infrared or near-infrared imaging, while other tests may require imaging under visible light, and/or a display screen to show content to the patient. However, ophthalmic testing devices such as a phoropter, autorefractor and photo-refractors, may only provide the capability to perform a limited range of tests. It would be advantageous to be able to screen for most vision problems and diseases using a single integrated device. Furthermore, due to the number of tests, it may be additional advantageous to be able to conduct a suite of test without repositioning the patient or recalibrating the machine. Current devices may require different distances to accurately conduct each test, resulting in movement of either the patient or the device between test and reducing the overall efficiency of the device.

The various examples of the present disclosure are directed toward overcoming one or more of the deficiencies noted above.

### SUMMARY

In an example of the present disclosure, the systems described herein relate to a vision screening device, including: a housing having an emission surface; a first emitter board disposed within the housing and including one or more light sources controllable by a processing device of the vision screening device; a reflector connected to the first emitter board and partially surrounding the one or more light sources, the reflector configured to reflect from the one or more light sources out of the emission surface; a polarizer coupled to the reflector at an output side of the reflector; a beam splitter positioned between the polarizer and the emission surface such that light from the reflector passes through the beam splitter and reflected light received through the emission surface is directed at an angle relative to an incident angle of the reflected light; an imaging system of the vision screening device configured to receive the reflected light from the beam splitter; and one or more computing systems configured to perform a vision screening evaluation based on image data received from the imaging system.

In some aspects, a system for a vision screening device includes: an emitter board having: a support; one or more LEDs arranged in an array on the support; a reflector having a output side and a connection side where the reflector is mounted to the support, the reflector partially surrounding the one or more LEDs and configured to reflect emitted light from the one or more LEDs in a columnar direction; a polarizer coupled to the reflector at the output side; a beam splitter positioned downstream of the polarizer such that light from the reflector passes through the beam splitter along an incident axis and reflected light received at the beam splitter is directed at an angle relative to the incident axis; and an imaging system configured to receive the reflected light from the beam splitter and produce image data.

### BRIEF DESCRIPTION OF THE DRAWINGS

Features of the present disclosure, its nature, and various advantages, may be more apparent upon consideration of the following detailed description, taken in conjunction with the accompanying drawings.

FIG. 1 illustrates a clinician using a vision screening device to perform one or more visual acuity exams for a patient, in accordance with one or more examples.

FIG. 2 illustrates an example vision screening device showing a clinician-facing display and interface, in accordance with one or more examples.

FIG. 3 illustrates a first perspective view of an example vision screening device, in accordance with one or more examples.

FIG. 4 illustrates a second perspective view of the example vision screening device of FIG. 3, in accordance with one or more examples.

FIG. 5 illustrates a perspective view of an example vision screening device with a patient facing cover removed, in accordance with one or more examples.

FIG. 6 illustrates a section view of an example vision screening device, in accordance with one or more examples.

FIG. 7 illustrates a section view of an example vision screening device, in accordance with one or more examples.

FIG. 8 illustrates a light management system of an example vision screening device for managing and directing light and reflections, in accordance with one or more examples.

FIG. 9 illustrates a side view of the light management system of FIG. 8, in accordance with one or more examples.

FIG. 10 illustrates an emitter for a light management system of an example vision screening device, in accordance with one or more examples.

FIG. 11A illustrates a side view of the emitter of FIG. 10, in accordance with one or more examples.

FIG. 11B illustrates a detail view of a portion of the emitter of FIG. 11A, in accordance with one or more examples.

FIG. 12 illustrates a system architecture of a vision screening device, in accordance with one or more examples.

FIG. 13 illustrates a system architecture of a vision screening system for performing vision screening tests and recording test result data for patients, in accordance with one or more examples.

FIG. 14 illustrates a side view of a light management system, in accordance with one or more examples.

### DETAILED DESCRIPTION

The present disclosure is directed to, in part, a vision screening device, and corresponding methods. Such an example vision screening device may be configured to perform one or more vision screening tests on a patient and to output the results of the vision screening test(s) to an operator of the device, such as a clinician or a physician's assistant. Specifically, the present disclosure is directed to devices and methods for managing light and reflections during a vision screening exam to improve performance of vision screening exams such as visual acuity exams.

The vision screening device described herein may include light and imaging channels for near infrared (NIR) as well as white light. The combination of the imaging channels provides additional functionality as well as further screening exams to be performed. Emitters for the NIR and the white light are mounted within a housing of the vision screening device and it is important for accurate results, when using the vision screening device, that stray light is prevented from reaching the imaging system of the vision screening device and interfering with the results of the exam. For example, stray white light from an environment may interfere with a test being performed using the vision screening device and may result in test results that are inaccurate or unreliable. Furthermore, stray light may result in image anomalies due to the unwanted light, as the imaging system of the vision screening device and associated processing components may be configured to generate image data for exams and evaluation in the absence of such anomalies (which may cause false detections or inaccuracies in the results of the exams).

The light emitters of the vision screening device, such as LEDs, are mounted within a housing of the vision screening device and structurally supported to provide positioning and alignment to an imaging system (e.g., an imaging path) for the vision screening device. Stray light control is needed to prevent light paths from interfering with each other and/or to prevent stray light from the environment form interfering with a designated light path.

In some examples, stray light may be controlled by means of absorbers such as filter glass or coatings or reflectors such as selective wavelength reflection. The control means may also include light traps and/or other systems for preventing light from reflecting within the housing of the vision screening device. Additionally, light returning from a test subject will also have to be controlled to ensure that once the reflected light enters the vision screening device, the light is directed to an intended destination (e.g., the imaging system) and not anywhere else within the housing. In addition, the ambient light from the environment also needs to be controlled to prevent image anomalies in the final results of the screening exams. Such anomalies or “ghost images” may be the result of ambient light, stray light, or uncontrolled reflected light. Because the LEDs are mounted within the housing of the vision screening device any stray light emitted by the LEDs must also be controlled to prevent interference and artifacts in images. The systems described here provide for light control systems within a vision screening device configured to perform visual acuity and other exams for patients.

The vision screening device described herein enables a clinician to perform a visual acuity assessment at a range of distances based on the space constraints and/or preferences of the individual. The vision screening device allows and accounts for the variation in distance while providing a consistent visual acuity result. Additionally, the vision screening device provides improvements for typical visual acuity workflows, for example by automatically presenting scrambled or rearranged orders of the digital optotypes to prevent patient memorization when moving from a first eye to a second eye.

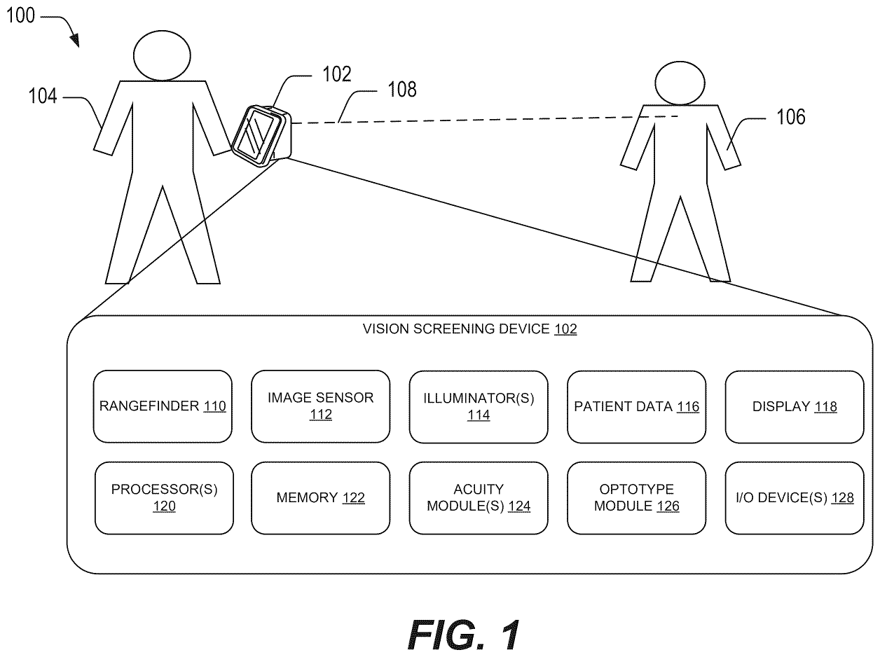

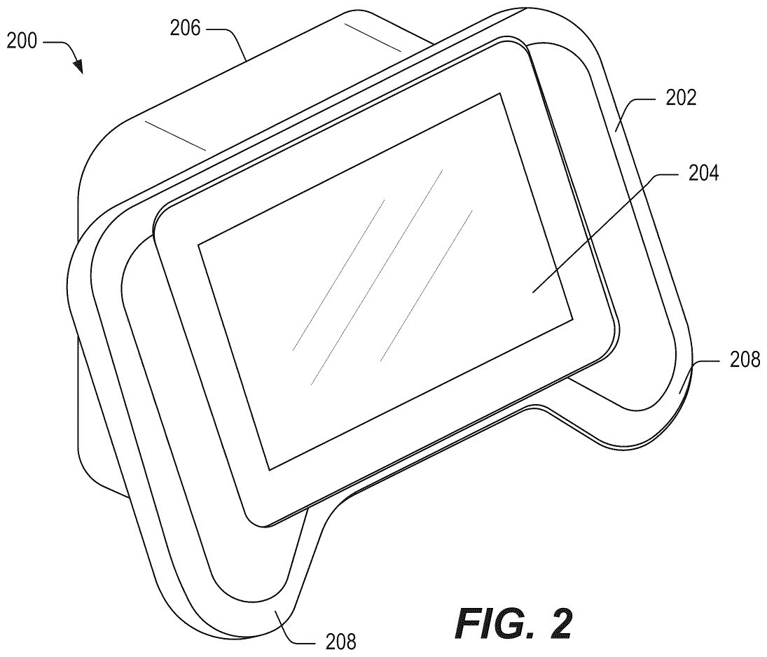

Turning now to the figures, FIG. 1 illustrates a system 100 with a clinician 104 using a vision screening device 102 to perform one or more visual acuity exams for a patient 106, in accordance with one or more examples. The clinician 104 may use the vision screening device 102, described in further detail with respect to FIGS. 2-13 below to perform the visual acuity exam among other exams for screening the patient 106 for one or more vision-based criteria. The vision screening device 102 may be used to perform the various exams on the patient 106 at a distance 108 from the patient. One or more different exams or screening processes may be performed on the patient 106. For example, a red reflex test, visual acuity exam, and other such tests may be performed on the patient 106 using the vision screening device 102. Visual acuity, and other exams may be assessed at various distances. Among some of the reasons for measuring at particular distances include assessor preferences and space limitations within an office. The vision screening device 102 described herein enables a clinician to perform a visual acuity exam and/or other exams at a range of distances based on the space constraints and/or preferences of the individual performing the exam. The vision screening device 102 allows and accounts for the variation in distance (e.g., to accurately provide optotypes of the correct size for a visual acuity exam) while providing a consistent visual acuity result. Additionally, the vision screening device 102 provides improvements for typical visual acuity workflows, for example by automatically presenting scrambled or rearranged orders of the digital optotypes to prevent patient memorization when moving from a first eye to a second eye.

The vision screening device 102 provides the clinician 104 with an option select a predetermined distance to the patient 106 for performing the visual acuity test. In an example, the vision screening device 102 gives the clinician 104 the option to select a 10-foot visual acuity test or a 5-foot visual acuity test. The 5-foot distance may allow the clinician 104 to perform all of the vision screening tests at a common distance, while the 10-foot distance may allow the clinician 104 to perform a visual acuity test at a more traditional distance. Once the clinician 104 selects the distance to use, the optotypes displayed on the vision screening device 102 are adjusted to a proper size for that respective distance.

The vision screening device 102 may be used for screening for diseases and abnormalities of the eyes of patients 106 as well as performing visual acuity exams for patients 106. In particular, the vision screening device 102 may include components for displaying optotypes, receiving inputs indicative of responses to the optotypes, generating recommendations for optotypes, capturing images of the eye(s) of the patient 106 under near-infrared as well as visible light radiation and conducting other ocular assessments such as mobility, color recognition, and/or visual acuity.

The vision screening device 102, shown and described in additional detail herein, at least with respect to FIGS. 2-13, includes a rangefinder 110, image sensor 112, illuminator(s) 114, storage for patient data 116, display 118, processor(s) 120, memory 122, acuity module(s) 124, optotype module 126, and I/O device(s) 128. The rangefinder 110 may include a distance sensor or other system for determining the distance 108 to the patient 106 from the vision screening device 102. The rangefinder 110 provides distance data that may be used to determine the spacing between the vision screening device 102 and the patient 106. The rangefinder 110 may provide distance data such that the distance 108 may be determined to within a range of less than a foot (e.g., having a tolerance of up to one foot).

The vision screening device 102 includes components for controlling the emission of both near-infrared and visible light radiation (e.g., the illuminator(s) 114) and the corresponding components such as the image sensor 112 configured for capturing the reflected radiation from the eye(s) of the patient 106 during the screening. In examples, near-infrared images may be captured by the image sensor 112 before initiating the capture of visible light images, so that pupils of the eyes(s) of the patient 106 do not constrict, or accommodate, during the screening in response to visible light, and the screening may be completed without the need for dilation of the eyes. The vision screening device 102 may further include components for analyzing the captured images to determine and/or aid in diagnosing diseases, conditions, and/or abnormalities in the eye(s) of the patient 106, and components for determining and reporting of output(s) indicating the disease conditions and/or abnormalities detected during the screening.

The storage for patient data 116 may include storage devices for storing results of the tests or examinations performed using the vision screening device 102. In some examples such storage may be implemented as part of the memory 122 of the vision screening device 102 and/or may also include cloud-based or remote storage. In some examples the patient data 116 for the exams performed by the vision screening device 102 may be transmitted to a remote computing system and/or stored on-board the device for later use and consumption, such as by adding or inputting into an electronic medical record of the patient 106.

The display 118 includes a display that faces towards the patient 106 from the vision screening device 102 and provide optotypes or other displays for use in a vision screening exam, for example to display a portion of an eye chart for a visual acuity exam. The display 118 may include any suitable display of digital information that can be used to project information towards the patient 106.

The processor(s) 120 and memory 122 may be used to perform one or more operations as part of a visual acuity exam or other vision screening exam. described herein. For example, the processor(s) 120 and memory 122 may be used to recommend optotypes for a visual acuity exam (e.g., through the optotype module 126) as well as to perform and/or aid in performance of the visual acuity exam (e.g., through the acuity module 124).

For example, the acuity module 124 may be used by the vision screening device 102 to provide the clinician 104 with an option select a distance 108 to the patient 106 for performing the visual acuity test. In an example, the vision screening device 102 gives the clinician 104 the option to select a 10-foot visual acuity test or a 5-foot visual acuity test. The 5-foot distance may allow the clinician 104 to perform all of the vision screening tests (e.g., including other tests such as a red reflex test or other such test that is performed at the 5-foot distance) at a common distance, while the 10-foot distance may allow the clinician 104 to perform a visual acuity test at a more traditional distance. Once the clinician 104 selects the distance to use, the optotypes displayed on the display 118 of the vision screening device 102 are adjusted to a proper size for that respective distance.

The vision screening device 102 may also use the rangefinder 110 to provide distance data and guidance for the clinician 104 to position the patient 106 and/or vision screening device 102 relative to one another such that the spacing meets the predetermined distance selected by the clinician 104. The rangefinder 110 and the distance data may be used to determine a distance and guidance may be displayed on a display of the vision screening device 102 instructing the clinician 104 to move closer to the patient 106 or further from the patient 106 until within a threshold distance of the predetermined distance, at which point the vision screening device 102 may begin the visual acuity exam using the acuity module 124. Furthermore, the vision screening device 102 may conduct each of the screening tests from a common distance as to remove the requirement of moving the patient 106 or recalibrating the vision screening device 102.

The display 118 on the vision screening device 102 may not be large enough to display an entire eye chart for a visual acuity exam. Accordingly, the vision screening device 102 may display only a portion of the eye chart such as a single line at a time or two lines at a time. To perform the visual acuity exam, the vision screening device 102 may display multiple different optotypes representing different lines of an eye chart to the patient to determine the visual acuity result for the patient 106.

In an illustrative example, the vision screening device 102 displays a critical line measurement (e.g., a line on a vision screening chart that represents a level of visual acuity that a person of a particular age group is expected to be able to read accurately). The clinician 104 may score or record the response of the patient 106 to the line. If the patient 106 passes, the vision screening device 102 automatically displays a next smaller line of characters. This process may then iteratively continue until the patient 106 fails a line. If the patient 106 fails the critical line, the vision screening device 102 automatically displays a next larger line of characters. This process may also continue until the patient 106 passes a line. In either scenario, the last line that a patient 106 passes represents that patient's vision. The vision screening device 102 may expedite the vision screening process for visual acuity exams by automatically displaying the next line for the patient based on their received response and therefore speeds up the workflow by reducing total number of button presses or interactions by a clinician.

In some examples the response from the patient 106 may be recorded by the clinician 104 marking on a display (e.g., the I/O device 128) of the vision screening device 102 whether the patient 106 correctly or incorrectly replied to each character or object. For instance, objects that are correct may be tapped while incorrect objects may be unmarked or vice versa. At the completion of the line the vision screening device 102 automatically proceeds to the next smaller or next larger line of the visual acuity chart based on the response of the patient 106.

In some examples the vision screening device 102 may automatically record the response of the patient 106 and score the response against the displayed optotype. For instance, the vision screening device 102 may include a microphone (e.g., as part of the I/O device 128) used to receive audio data as the patient 106 reads the displayed optotype and may process the audio data to determine the response (e.g., by using a transcription method or other such technique) and then comparing the received response against the optotype before proceeding to the next line of the visual acuity exam.

Based at least in part on the on analysis of the captured images by the vision screening device 102 and the received acuity data, the vision screening device 102 may generate an output including at least one of a recommendation or a diagnosis associated with the patient. Such an output (e.g., the recommendation and/or the diagnosis) may be indicative of the visual acuity of one or both of the eyes of a patient 106. For example, the device may be used by a clinician 104 to determine a visual acuity score for a left eye and for a right eye.

As the clinician moves from scoring the left eye to the right eye of the patient, the optotype module 126 may cause the optotypes to be rearranged such that the characters initially presented in a first optotype at a first size for the right eye are presented in a second optotype at the first size for the left eye (or vice versa) but in a different order or arrangement. This may prevent a patient from memorizing the line during screening of a first eye and thereby lead to more accurate visual acuity results.

In addition to providing efficiencies for clinicians by automatically proceeding to a subsequent displayed optotype, the vision screening device 102 may be used to determine and/or recommend an optotype or set of optotypes to present to the patient for the visual acuity exam through the optotype module 126. For instance, the optotype module 126 may receive factors such as the patient age (e.g., and whether they are expected to be able to read letters), patient language, and/or geographic region may be used to recommend or determine a set of optotypes to present to the clinician 104 for selection and use in the screening. The geographic information may be input by the clinician 104 without requiring any additional patient information and may suggest a set of optotypes based on a language spoken by a majority of the population in the geographic region, or may provide the clinician 104 with one or more options to select from based on the current region. After the geographic region is determined, the clinician 104 may also input the patient age or other patient characteristic data (which may automatically be accessed from an electronic medical record if such data is available for access by the vision screening device, such as when in a clinician office storing or having access to such a patient record. The optotype module 126 may then recommend or determine an optotype to present based on the provided data. The optotype module 126 may present a recommendation for confirmation by the clinician or may automatically present the optotype, which may be changed or reconfigured by the clinician. The different optotypes may include different alphabet or language options as well as options for different optotypes such as Snellen charts, lea symbol charts, tumbling E charts, and other such optotypes.

In this manner, the vision screening device provides immediate access to a recommended optotype to use for each patient and that recommended optotype can then be displayed to the patient digitally. This allows the clinician to provide the best possible assessment for that particular patient, and speeds up workflow as the clinician does not have to sort through manual charts to find a proper one to display to the patient (if they even have the proper chart as some clinician resources or offices may be limited to only a small set of charts).

Furthermore, the device may incorporate components, such as I/O device(s) 128, which may be configured to display images or graphics associated with visual acuity, mobility, and/or color recognition tests to the clinician 104 and also provides for the clinician 104 to interact with the vision screening device 102.

Additional details pertaining to the above-mentioned devices and techniques are described below with reference to the following figures. It is to be appreciated that while these figures describe devices and systems that may utilize the claimed methods, the methods, processes, functions, operations, and/or techniques described herein may apply equally to other devices, systems, and the like.

FIG. 2 illustrates a vision screening device 200 showing a clinician-facing display and interface, in accordance with one or more examples. The vision screening device 200 is illustrated with a housing 202 that encloses the components described below. The housing 202 is depicted with protrusions 208 that may enable a user to grasp the edges of the vision screening device 200 during use.

As illustrated in FIG. 2, in some examples an operator may administer vision screening tests, via a vision screening device 200, on a patient to determine eye health of the patient. As described herein, the vision screening device 200 may perform one or more vision screening tests, including screening for diseases and/or abnormalities of eye(s) when the eyes are illuminated by visible light. In addition, the vision screening device 200 may also be configured to perform other vision screening tests, such as a visual acuity test, a refractive error test, an accommodation test, dynamic eye tracking tests, color vision screening test and/or any other vision screening tests, configured to evaluate and/or diagnose the vision health of the patient. In examples, the vision screening device 200 may comprise a portable device configured to perform the one or more vision screening tests. Due to its portable nature, the vision screening device 200 may perform the vision screening tests at any location, from conventional screening environments, such as schools and medical clinics, to physician's offices, hospitals, eye care facilities, and/or other remote and/or mobile locations. Furthermore, the vision screening tests may be conducted at a common distance, providing for a more efficient process and resulting in the vision screening device 200 being capable of being used for large group testing. It is also envisioned that the vision screening device 200 may be used for administering vision screening tests to all age groups, including newborns and young children and geriatric patients.

As described herein, the vision screening device 200 may be configured to perform one or more vision screening tests on the patient. In examples, one or more vision screening tests may include illuminating the eye(s) of the patient with infrared or near-infrared (NIR) radiation and capturing reflected radiation from the eye(s) of the patient. For example, U.S. Pat. No. 9,237,846, the entire disclosure of which is incorporated herein by reference, describes systems and methods for determining refractive error based on photorefraction using pupil images captured under different illumination patterns generated by near-infrared (NIR) radiation sources. In other examples, vision screening tests, such as the red reflex test, may include illuminating the eye(s) of the patient with visible light, and capturing color image(s) of the eye(s) under visible light illumination. The vision screening device 200 may acquire data comprising color images and/or video data of the eye(s) under visible light illumination, and detect pupils, retinas, and/or lenses of the eye(s) of the patient. This data may be used to determine differences between left and right eyes, compare the captured images with standard images, or generate visualizations to assist the operator or a clinician in diagnosing diseases and abnormalities of the eye(s) of the patient. The vision screening device 200 may transmit the data to a vision screening system for analysis to determine an output associated with the patient. Alternatively, or in addition, the vision screening device 200 may perform some or all of the analysis locally to determine the output.

Indeed, in any of the examples described herein, some or all of the disclosed methods may be performed in whole or in part by the vision screening device 200 independently (e.g., without the vision screening system or its components), or by the vision screening system independently (e.g., without the vision screening device 200 or its components). For instance, in some examples, the vision screening device 200 may be configured to perform any of the vision screening tests, and/or other methods described herein without being connected to, or otherwise in communication with, the vision screening system. In some examples, the vision screening system may include one or more components that are similar to and/or the same as those included in the vision screening device 200, and thus, the vision screening system may be configured to perform any of the vision screening tests, and/or other methods described herein without being connected to, or otherwise in communication with, the vision screening device 200.

The vision screening device 200 may include one or more radiation source(s) (not shown in FIG. 2) configured to perform functions associated with administering one or more vision screening tests. The radiation source(s) may comprise individual radiation emitters, such as light-emitting diodes (LEDs), which may be arranged in a pattern to form an LED array. In examples, the radiation source(s) may include near-infrared (NIR) radiation emitters, such as NIR LEDs, for measuring the refractive error of the eye(s) of the patient using photorefraction methods. The NIR radiation emitters of the radiation source(s) may also be used for measuring the gaze angle or gaze direction of the eye(s) of the patient. In addition, the radiation source(s) may also include color LEDs for generating color stimuli for display to the patient during a color vision screening test.

The vision screening device 200 may also include one or more radiation sensor(s), such as infrared cameras, configured to capture reflected radiation from the eye(s) of the patient during the vision screening test(s). For example, the vision screening device 200 may emit, via the radiation source(s), one or more beams of radiation, and may be configured to direct such beams at the eye(s) of the patient. The vision screening device 200 may then capture, via the radiation sensor(s), corresponding radiation that is reflected back (e.g., from pupils of the eye(s)). In examples, the radiation sensor(s) may comprise NIR radiation sensor(s) to capture reflected NIR radiation while the eye(s) of the patient are illuminated by the NIR radiation source(s). The data captured by the NIR radiation sensor(s) may be used in the measurement of the refractive error and/or gaze angle(s) of the eye(s) of the patient. The data may include images and/or video of the pupils, retinas, and/or lenses of the eyes of the patient. In some examples, the images and/or video may be in grayscale (e.g., with values between 0 and 128, or between 0 and 256). The data may be captured intermittently, during specific periods of the vision screening test(s), or during the entire duration of the test(s). Additionally, the vision screening device 200 may process the image(s) and/or video data to determine change(s) in the refractive error and/or gaze angle(s) of the eye(s) of the patient. The grayscale images of the eye(s) captured under NIR illumination may also be used for screening for diseases and abnormalities of the eye(s) such as ametropia, strabismus, and occlusions.

In examples, the vision screening device 200 may further include visible white light source(s) and a camera configured to capture color images and/or video of the eyes under illumination by the white light source(s). The white light source(s) may comprise light-emitting diodes (LEDs) such as an array of LEDs configured to produce white light e.g., a blue LED with a phosphor coating to convert blue light to white light, or a combination of red, blue, and green LEDs configured to produce white light by varying intensities of individual red, blue and green LED activation. Individual LEDs of the array of LEDs may be arranged in a pattern configured to be individually operable to provide illumination from different angles during the vision screening test(s). The white light source(s) may also be configured to produce white light of different intensity levels. The camera may be configured to capture white light reflected from the eyes of the patient to produce digital color images and/or video. In some examples, pixel values in the color images and/or video may be in a RGB (red, green, blue) color space. The color images and/or video of the eye(s) captured under white light illumination may be used for screening for diseases and abnormalities of the eye(s) such as cataracts, media opacities in aqueous and vitreous humors, tumors, retinal cancers and detachment, and the like. In addition, the color images and/or video may be used in conjunction with the grayscale images captured under NIR illumination to generate visualizations to assist in the detection of a wide range of disease conditions of the eye(s).

The vision screening device 200 may also include one or more display screen(s), such as display 204, which may be color LCD (liquid crystal display), or OLED (organic light-emitting diode) display screens. The display 204 may be an operator display screen facing a direction towards the operator, configured to provide information related to the vision screening tests to the operator. In any of the examples described herein, the display 204 facing the operator may be configured to display and/or otherwise provide the output generated by the vision screening device 200 and/or generated by the vision screening system. The output may include testing parameters, current status and progress of the screening test(s), measurements(s) determined during the test(s), image(s) captured or generated during the screening test(s), a diagnosis determined based on one or more tests, and/or a recommendation associated with the diagnosis. The display 204 facing the operator may also display information related to or unique to the patient, and the patient's medical history.

In some examples, the vision screening device 200 may also include a display screen (not shown in FIG. 2) facing in a direction towards the patient and configured to display content to the patient. The content may include attention-attracting images and/or video to attract attention of the patient and hold the patient's gaze towards the vision screening device 200. Content corresponding to various vision screening test(s) may also be presented to the patient on the display screen. For example, the display screen may display color stimuli to the patient during a color vision screening test, or a Snellen eye chart during a visual acuity screening test. The display screens may be integrated with the vision screening device 200, or may be external to the device, and under computer program control of the vision screening device 200.

The vision screening device 200 includes additional sensors within the housing 202 including a distance sensor such as a range finder, an ambient light sensor, ambient infrared sensor, and other such sensors and components as described herein and as may be used by a clinician during a vision screening exam.

Depicted in FIG. 2, the vision screening device 200 includes a display 204 that may be used by a clinician or other user to interact with the vision screening device 200. The display 204 may be used for inputting information related to a patient, selecting test parameters, adding additional evaluation inputs, and otherwise controlling the vision screening device 200. As depicted, the display 204 may be a touch-screen or other similar display that enables user input through the display 204 while also providing output to the clinician.

On a side of the housing 202 opposite from the display 204 is a patient-facing surface 206. The patient-facing surface 206 includes an emission surface through which light and other radiation sources are emitted towards a patient during an exam. Additionally, the patient-facing surface 206 enables a distance sensor enclosed within the housing (not shown in FIG. 2) to gather distance data for use in positioning the vision screening device 200 relative to a patient for evaluation.

The vision screening device 200 may be used for various evaluations at prescribed distances for distance vision testing. For example, vision testing and evaluation may be performed at a distance of five feet from the patient. The distance between the patient and the vision screening device is important for photorefraction exams (typically performed at a distance of three feet in previous systems) as well as visual acuity tests (typically tested at distances between ten and twenty feet). The vision screening device 200 is designed to perform the various tests and evaluation at a common distance for ease and speed of testing. The single common distance enables simpler exam processes as patients need not be shifted for different tests. Additional vision tests such as color vision and near vision may also be performed at the common distance (e.g., five feet or ten feet). Further still, to aid in vision screening, the vision screening device may be programmed to perform a sequence of exams simultaneously or back-to-back without requiring a reset or changing parameters of the vision screening device 200. In some examples, the clinician may use the vision screening device 200 to perform a suite of tests, or may select a subset of tests to perform during an evaluation.

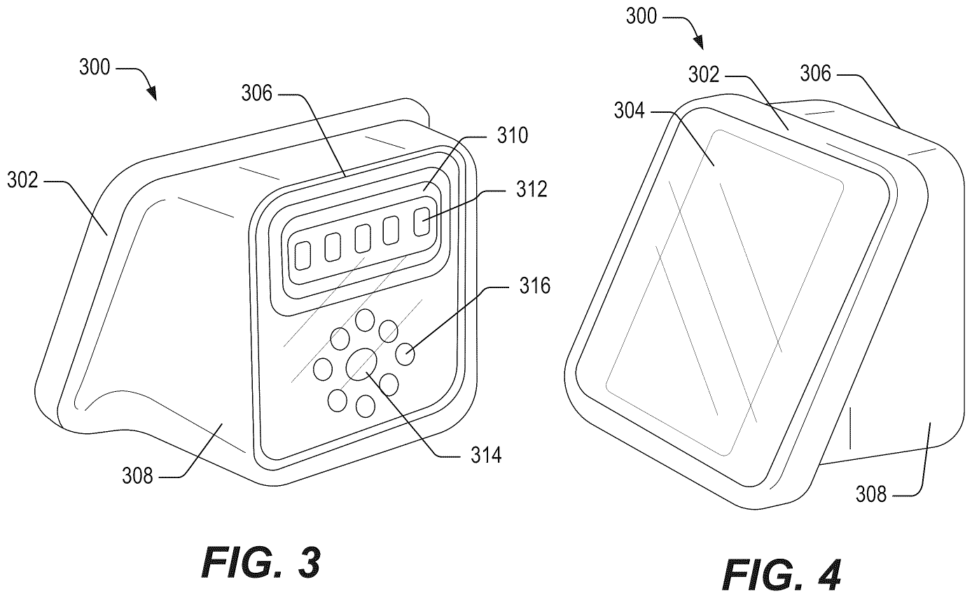

FIGS. 3-4 illustrate perspective views of an example vision screening device 300, in accordance with one or more examples. The example vision screening device 300 includes a housing 302 similar or identical to the housing 202. Additionally, the vision screening device 300 includes a display 304 similar or identical to the display 204 and an emission surface 306 on a patient-facing side. The emission surface 306 provides a surface through which light and/or other emissions may be projected towards a patient and also enables reflected light and other signals to be received. The housing 302 encloses a space 308 that receives reflected light and NIR light after it reflects off the patient. The emission surface 306 may be transparent to such light and signal transmissions, or include transparent portions, for example around a second display 310 and emitters 314 and aperture 316.

The second display 310 faces the patient and is used to display digital objects 312 such as letters, images, shapes, and other such digital display objects. The second display 310 may display the various digital objects 312 at different sizes or heights, for example to test visual acuity based on digital objects 312 of decreasing height. The second display 310 transmits digital information outwards towards the patient for the patient to interact with (e.g., read) while the emitters 314 and aperture 316 are used to gather information regarding the eyes of the patient.

The emitters 314 and aperture 316 are used to emit NIR light and white light towards the patient and receive reflected light off the patient at the aperture 316. The reflected light passes through the aperture 316 and into the interior of the housing 302 where it is reflected to a sensor (e.g., camera) or multiple cameras that may be used to detect the white light and NIR reflection data.

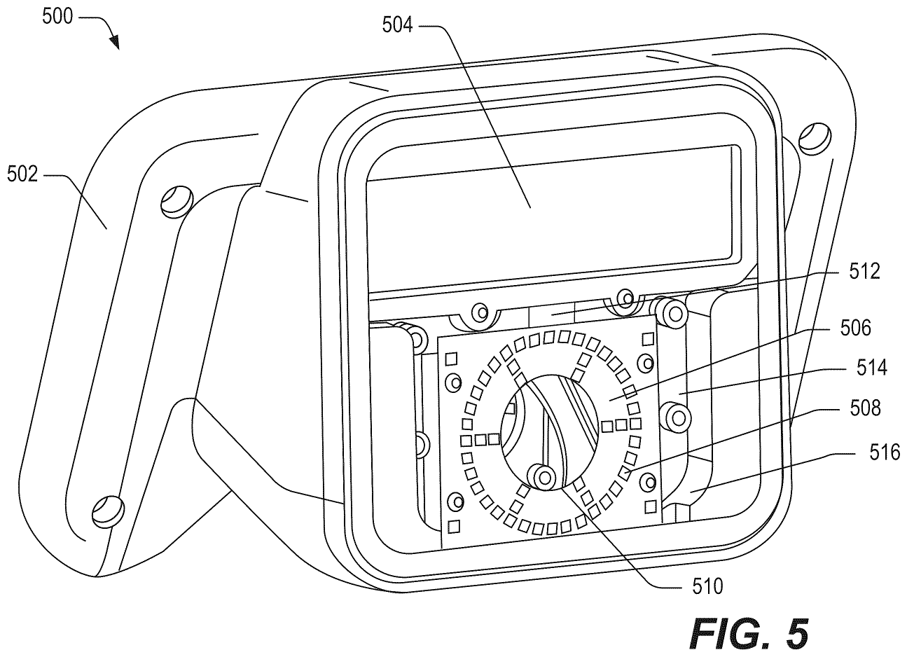

FIG. 5 illustrates a perspective view of an example vision screening device 500 with a patient facing cover removed, showing a partial view of internal components stored within a housing 502 of the vision screening device 500, in accordance with one or more examples. In addition to the housing 502, the vision screening device 500 includes a display 504 which serves as a patient-facing display to display digital objects such as symbols, pictures, letters, and other such information to a patient during an exam.

With the cover removed, an interior of the housing 502 is depicted including an emitting board 506 that includes emitters 508. The emitting board 506 provides power from a power source of the vision screening device 500 to the emitters 508 and also provides for a processor of the vision screening device 500 to control the emitters 508 to selectively emit radiation towards the patient. The emitters 508 may include NIR emitters as well as visible light emitters (e.g., white light emitters) that project light out of the patient-facing side of the vision screening device 500 towards the patient. The emitters 508 may include LEDs and other sources of near infrared light as well as visible light or other types of emitted radiation that may be directed towards the patient and received back at the vision screening device 500 after reflecting off the patient.

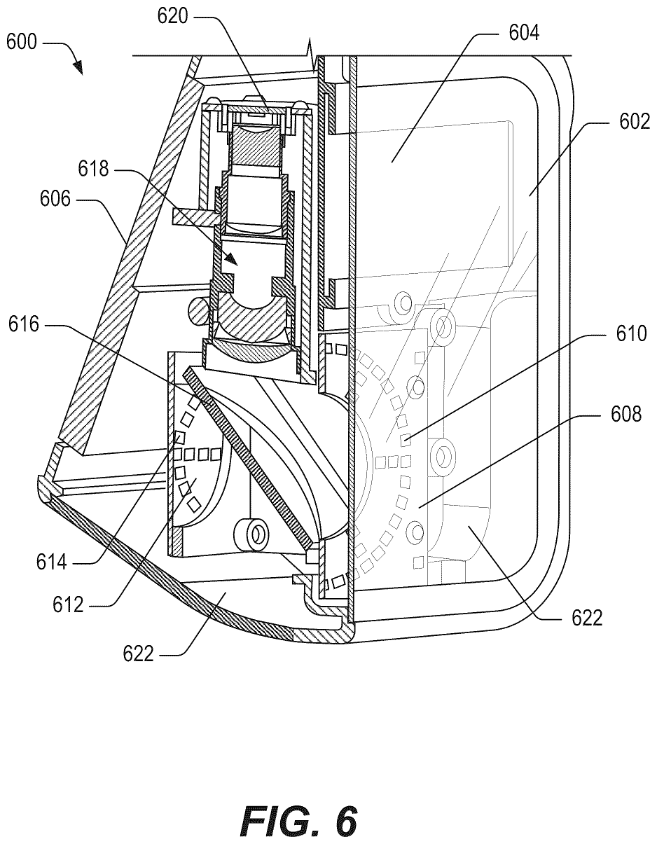

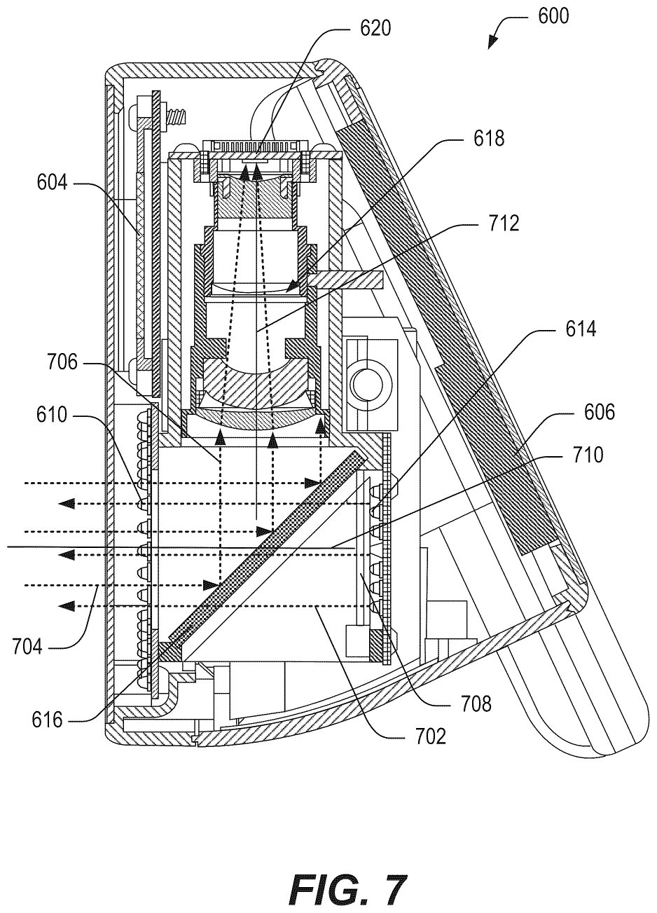

The emitting board 506 defines an opening 510 at a center of the array of the emitters 508. The array of emitters 508 may share an optical axis and may direct light or radiate energy outwards toward the patient along near parallel directions. The reflected energy from the patient passes through the opening 510 to reach further internal components of the vision screening device 500 for sensing and detection, as depicted in FIGS. 6-7.

The vision screening device 500 further includes a distance sensor 512. In FIG. 5 the distance sensor 512 is depicted positioned facing the patient and disposed vertically underneath the display 504. In some examples, the distance sensor 512 may be positioned within the housing 502 an any suitable position facing the patient-side of the housing 502 and oriented to face the patient when in use. The distance sensor 512 may include an ultrasonic sensor (and be positioned at the rear surface of the housing 502 with no glass between the distance sensor 512 and the patient, a millimeter wave radar sensor, a light-base sensor, or other such distance measuring sensor.

The vision screening device 500 is shown with a rigid chassis 514 that floats within the housing 502. The optical components of the vision screening device including the emitters, beam splitters, lenses, and filters are aligned and calibrated and secured to the rigid chassis 514. In examples, the emitters of the LED boards, lenses, image sensors are all aligned and centered for capturing image data. The rigid chassis 514 floats within the housing, and may be mounted to the housing through one or more shock absorbers, energy absorbing devices, and other such components to provide protection for the components in the event the vision screening device 500 is dropped. In examples, one or more of the components, such as the clinician-facing display and/or patient-facing display may be surface mounted to a skin of the housing 502. The housing 502 further includes one or more access ports for servicing the interior components, for example to calibrate or re-align the optical components. The vision screening device 500 also includes anti-reflective coatings 516 such as light traps, beam dumps, non-reflective paint, matte solder masks, and the like.

FIGS. 6-7 illustrate a section view of an example vision screening device 600, in accordance with one or more examples. In the section view, the vision screening device 600 is shown with elements of the vision screening device shown and described with respect to FIGS. 2-5 above, including a housing 602, display 604, display 606, emitting board 608, and emitters 610. The section view of FIG. 6 illustrates aspects of the interior of the vision screening device including a second emitting board 612, emitters 614, beam splitter 616, lens and filter assembly 618, and sensor 620.

Within the vision screening device 600, and positioned behind the emitting board 608 is a beam splitter 616 that is used to split light based on the direction from which the light is incident. Light reaching the beam splitter 616 from within the housing (e.g., behind the beam splitter 616) that is emitted by the emitters 614 of the second emitting board 612, is transmitted through a polarizer (not shown in FIG. 6) and then through the beam splitter and remains traveling in a direction parallel or substantially parallel with the direction or incident angle of the incoming light. The polarizer may be positioned between the beam splitter and the second emitting board 612. In contrast, reflected light that reaches the beam splitter 616 from the environment (and passes through an emitting surface of the vision screening device 600) is reflected by the beam splitter towards the lens and filter assembly 618 and finally to the sensor 620 where the reflected light is received and subsequently detected and processed as described herein. In some examples, the beam splitter 616 may be a plate beam splitter with a coating that causes the incident light originating from outside the vision screening device 600 to be reflected upwards (as oriented in FIG. 6) and into the lens and filter assembly 618. In examples, the beam splitter 616 may be accompanied with one or more additional filters or elements in the light path of the emitted NIR and/or visible light. The filter may, for example be a polarizing filter that polarizes the light as it is emitted from the vision screening device (e.g., after passing through the beam splitter from the emitters 614) and when the reflected light is returned, the beam splitter 616 may reflect the polarized light into the lens and filter assembly 618.

The lens and filter assembly 618 may include various filters including polarizing filters, filters to remove particular types of light, notch filters (e.g., to remove a portion of red light or other such light ranges), or otherwise treat the visible light and/or NIR light as it travels to the sensor 620. In examples, the filters may include coatings disposed on one or more lenses of the lens and filter assembly 618. The sensor 620 may include one or more sensors, such as a first sensor that detects NIR electromagnetic radiation and a second sensor that detects visible light. In examples, the sensor 620 may be a single sensor equipped to detect both NIR electromagnetic radiation as well as visible light. The light received at the sensor 620 may be processed based on the particular type of exam being performed either by an on-board processor and/or an external processing system to provide an output that may be displayed at the display 606 and/or output to an external system such as a system that hosts an electronic medical record for the patient.

Within the housing of the vision screening device 600, the interior surfaces 622 may be coated with anti-reflective materials and/or coatings. In some examples the coverings of the interior surfaces 622 may include beam dumps, light traps, matte paints and surface materials, and other such coatings or surface treatments that may prevent or resist reflection of light about the interior of the housing.

FIG. 7 illustrates a section view of the vision screening device 600 and is illustrated including light traces of emitted radiation and received reflected radiation. Emitted light 702 includes light from emitters 614 that extends along a first optical axis 710 including along a direction parallel to the first optical axis 710 and may include visible night, NIR, white light, blue light, red light, green light, or other such light beams as emitted from the emitters 614. The emitted light 702 passes from the emitters 614 through a polarizer 708 and then through the beam splitter 616. The emitted light then passes out of the housing 602. Additional emitted light emanates from emitters 610 and joins the emitted light 702 traveling parallel with the first optical axis 710. After reflecting off an environment, including the patient, reflected light 704 is returned to the housing 602. The reflected light 704 may come in through an aperture in the housing 602 and may be parallel to the first optical axis but in a direction opposite the emitted light 702 then reaches the beam splitter 616 and some or all of the reflected light 704 is reflected to a second optical axis 712 perpendicular to the first optical axis 710 as diverted light 706. Examples, the diverted light 706 may include a portion of the reflected light 704. In examples the portion may be in a range of ten percent to one hundred percent. The second optical axis 712 enables the system to receive and process the diverted light 706 at the sensor 620 and provides for a greater distance over which the reflected light 704 and diverted light 706 may be conditioned through lenses and filters before reaching the sensor 620. The additional distance is enabled within a housing that maintains a compact handheld footprint that remains thing and similar in shape and form factor to a tablet.

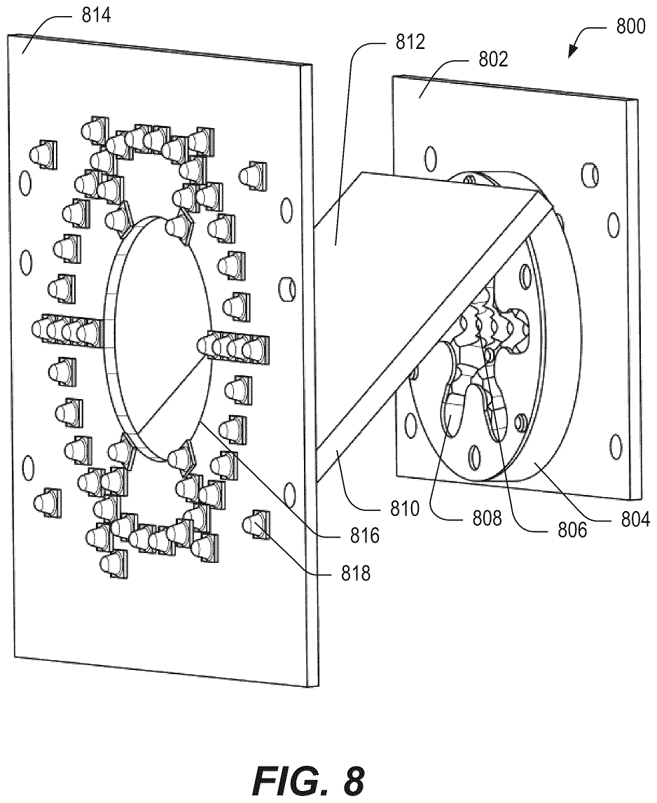

FIG. 8 illustrates a light management system 800 of an example vision screening device for managing and directing light and reflections, in accordance with one or more examples. The light management system 800 may be implemented in a vision screening device such as the vision screening device shown and described herein, for example with respect to FIGS. 1-7.

The vision screening device described herein may include light and imaging channels for near infrared (NIR) as well as white light. For example, the light management system 800 may include LEDs 806 and 818 configured to emit NIR as well as visible or white light. The combination of the imaging channels provides additional functionality as well as further screening exams to be performed. The LEDs 806 and 818 are mounted within a housing of the vision screening device and it is important for accurate results, when using the vision screening device, that stray light is prevented from reaching the imaging system of the vision screening device and interfering with the results of the exam. Stray light may result in image anomalies due to the unwanted light, as the imaging system of the vision screening device and associated processing components may be configured to generate image data for exams and evaluation in the absence of such anomalies (which may cause false detections or inaccuracies in the results of the exams).

The LEDs 806 and 818 are mounted within a housing of the vision screening device and structurally supported on a first emitter board 802 and a second emitter board 814 to provide positioning and alignment to an imaging system (e.g., an imaging path) for the vision screening device. Stray light control is needed to prevent light paths from interfering with each other and/or to prevent stray light from the environment form interfering with a designated light path.

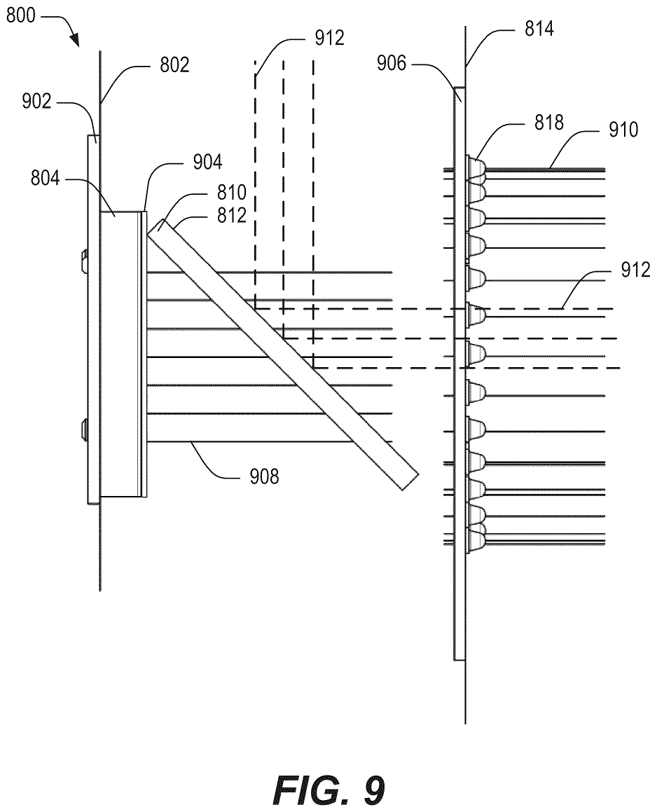

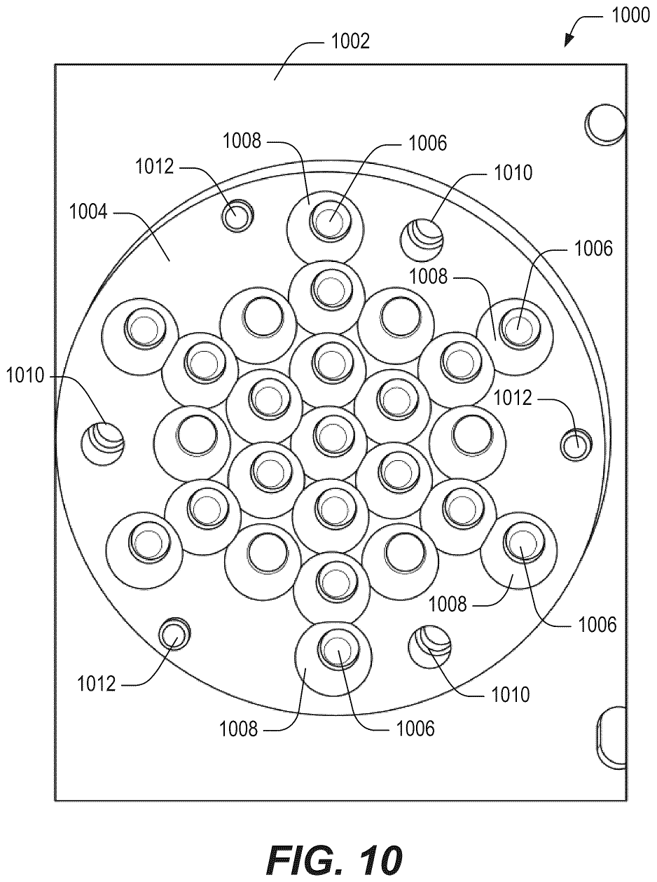

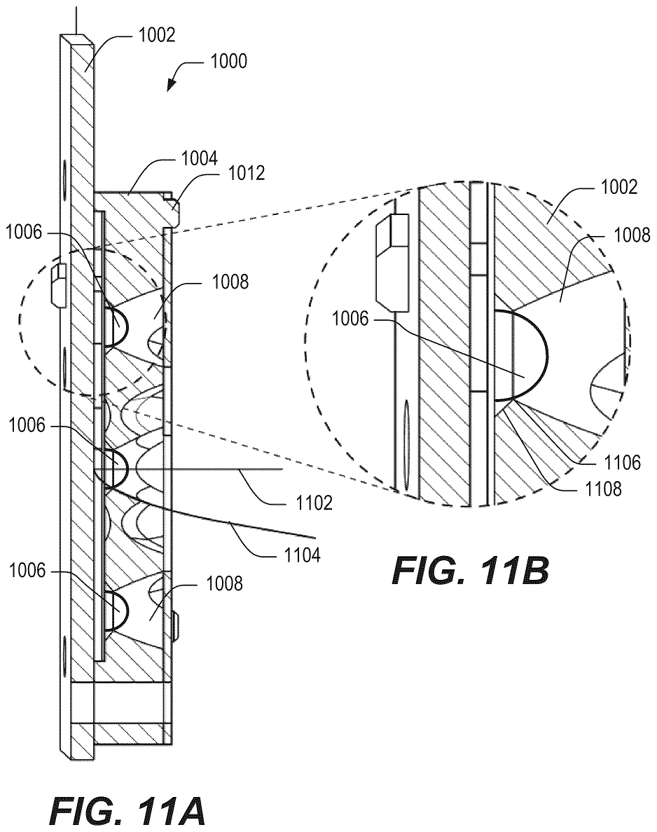

The light management system 800 includes a first emitter board 802 with LEDs 806 at least partially surrounded by a reflector 804, the reflector having individual reflecting surfaces 808 about each LED 806 to reflect light emitted by the LEDs 806. The LEDs 806 emit light in all directions and therefore would direct light into the housing that may then bounce or reflect around inside the housing and potentially cause anomalies or other errors during imaging of a patient. Accordingly, the reflector 804 is shaped and positioned to individually surround each of the LEDs 806 and reflect the light from the LEDs 806 into columnar light to proceed parallel with a light axis and exit the housing of the vision screening device. Additional details and geometry of the reflector 804 are shown and described with respect to FIGS. 10 and 11A-11B. Adjacent the reflector 804 is a polarizer (shown in FIG. 9) that polarizes the emitted light. The polarizer 804 may cover some or all of the LEDs on the emitter board. In examples, the first emitter board 802 and other components within the housing of the vision screening device may be coated with a non-reflective coating, such as a non-reflective paint. The electrical connections, such as solder connections for the LEDs 806 may be coated with a matte solder mask on the various boards. Additionally, one or more beam dumps or light traps may be situated within the housing to capture stray light and prevent it from bouncing and reflecting about within the housing.

After the light emitted from the LEDs 806 is reflected by the reflector 804 into columnar light, the columnar light passes through a beam splitter 810 positioned downstream of the reflector 804 and a polarizer 904 (shown in FIG. 9). The beam splitter 810 splits a beam of light into a transmitted and a reflected beam. The beam splitter 810 allows the columnar light to pass from the reflector 804 through the beam splitter 810 towards an emission surface of the vision screening device. The beam splitter 810 allows the polarized light to pass through the beam splitter 810 while reflected light from the environment (e.g., reflected polarized light) is reflected by the surface 812 of the beam splitter into an imaging system of the vision screening device. The beam splitter 810 may be configured to enable light of a first polarization to pass along a light axis while light of a second polarization is reflected to be parallel with a second axis different from the light axis. In some examples the second axis may be perpendicular to the light axis, such as shown in FIG. 9.

The light management system 800 includes a second emitter board 814 positioned adjacent the emission surface of the vision screening device within the housing, the second emitter board 814 supporting emitters such as LEDs 818. The LEDs 806 and 818 may include NIR and visible light emitters. The second emitter board 814 also defines an opening 816 that allows light from the first emitter board 802 to pass through to the emission surface of the vision screening device and out towards a patient. The opening 816 also allows reflected light to enter the housing and be incident upon the surface 812 of the beam splitter 810. In some examples the second emitter board 814 may include a reflector similar to the reflector 804 to reflect light from the LEDs 818 into columnar light directed towards the patient.

FIG. 9 illustrates a side view of the light management system 800 of FIG. 8, in accordance with one or more examples. The light management system 800 is shown with the components from FIG. 8, including the first emitter board 802, reflector 804, beam splitter 810, surface 812, second emitter board 814, and LEDs 818. The light management system 800 is illustrated with lines indicated directions for emitted light. The light management system 800 includes a mounting board 902 that supports and/or provides power and/or communication connections to the first emitter board 802 and the LEDs 806 positioned thereon. The polarizer 904 is also shown positioned against the output surface of the reflector 804. The polarizer 904 may cover some or all of the LEDs on the emitter board. The light is therefore emitted from the LEDs 806, reflected by the reflector 804 into columnar light 908 that passes through the polarizer 904 to produce a column of light having a first polarization and then passes through the opening 816 of the second emitter board 814. Additional light is emitted 910 by the LEDs 818 towards the patient.

Reflected light 912 is received at the vision screening device and passes through the opening 816 of the second emitter board. The reflected light 912 passes through the opening 816 of the second emitter board 814 and reflects off the surface 812 of the beam splitter 810 and the reflected light 912 is then directed into an imaging system of the vision screening device such as a series of lenses, filters, and imaging devices.

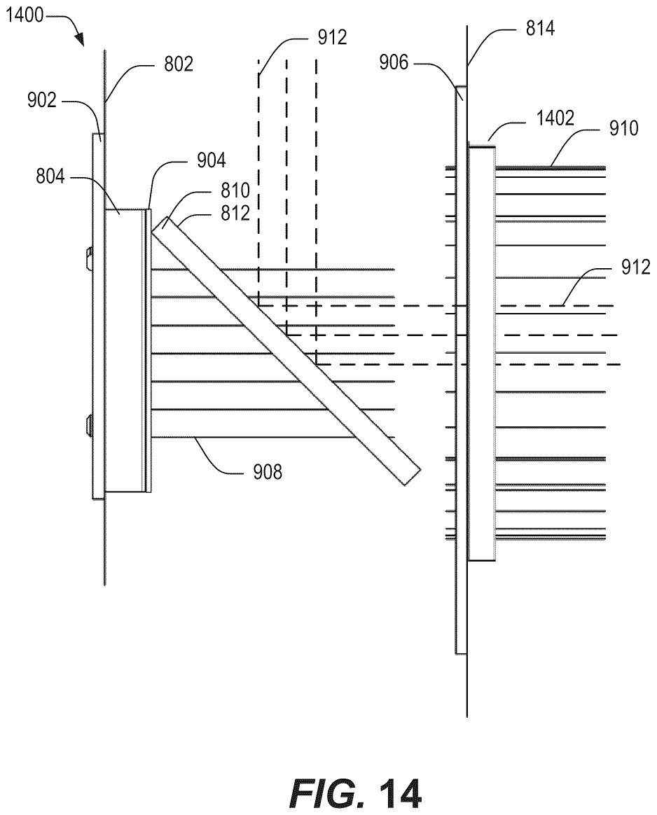

Turning now to FIG. 14, which illustrates a side view of a light management system 1400, in accordance with one or more examples. The light management system 1400 may be an example of the light management system 800 of FIG. 8, and includes components such as the first emitter board 802, reflector 804, beam splitter 810, surface 812, second emitter board 814, and LEDs 818. The light management system 1400 is illustrated with lines indicated directions for emitted light. The light management system 1400 includes a mounting board 902 that supports and/or provides power and/or communication connections to the first emitter board 802 and the LEDs 806 positioned thereon. The polarizer 904 is also shown positioned against the output surface of the reflector 804. The light is therefore emitted from the LEDs 806, reflected by the reflector 804 into columnar light 908 that passes through the polarizer 904 to produce a column of light having a first polarization and then passes through the opening 816 of the second emitter board 814. Additional light is emitted 910 by the LEDs 818 towards the patient.

In the example shown in FIG. 14, the light management system 1400 includes a reflector 1402 that may be similar or identical to the reflector 804 as shown and described herein. In some examples, the reflector 1402 may include a polarizer at an outlet side of the reflector 1402 similar to the polarizer 904. In some examples, the reflector 1402 may not include such a polarizer. The addition of the reflector 1402 to the LEDs 818 may aid in guiding light towards the patient, and may also reduce stray light reflections from the second emitter board 814 and LEDs 818 into the enclosure of the vision screening device (for example reflecting off a viewing window of the vision screening device).

FIG. 10 illustrates an emitter board 1002 for a light management system 1000 of an example vision screening device, in accordance with one or more examples. The light management system 1000 may be an example of a portion of the light management system 800 shown and described with respect to FIGS. 8-9. The light management system 100 may include an emitter board 1002 which may be similar or identical to the first emitter board 802 and a reflector 1004 similar or identical to the reflector 804. The light management system 1000 also includes LEDs 1006 that may include NIR and visible light LEDs.

The reflector 1004 may be formed of a material such as aluminum which may have a coating applied thereto, such as a non-reflective coating including a paint or anodization. The reflector may then be machined or polished at recesses 1008 to provide for reflection of light emitted from the LEDs 1006 without providing a reflective surface that may cause stray light or light reflections within the housing of the vision screening device. The recesses 1008 are arrayed at the locations of each of the LEDs 1006 such that each LED 1006 is situated within a respective recess 1008. The shape and structure of the recesses are shown and described in greater detail with respect to FIGS. 11A-11B herein. The recesses 1008 may be formed by machining into the material forming the reflector 1004 such as aluminum and therefore the flat surfaces may be coated with a non-reflective material while the recesses 1008 are reflective. The recesses 1008 may be subsequently polished to provide for reflection of the light emitted from the LEDs 1006.

The reflector 1004 being formed of a material such as aluminum also provides an opportunity for the reflector 1004 to serve as a heat sink for thermal management of the LEDs and/or electronic components of the vision screening device. The reflector 1004 may also include heat sink features such as fins and/or be connected to the emitter board 1002 through a thermally conductive material such as a thermal paste to enable the reflector 1004 to act as a heat sink and to dissipate heat from the components of the vision screening device.

The reflector 1004 further includes mounting holed 1010 for securing the reflector 1004 to the emitter board 1002 through the use of threaded connectors or other such fasteners. Through the reflector 1004 is shown with a circular perimeter or profile, other shapes and configurations for the reflector 1004 may be used and will be appreciated by those with skill in the art. Additionally, the array and arrangement of the LEDs 1006 and recesses 1008 may vary based on the particular arrangement needed for a use case and may be adjusted as will be understood by those with skill in the art.

The reflector 1004 also includes mounts 1012 disposed adjacent a perimeter of the reflector 1004. The mounts 1012 are disposed in an uneven arrangement, e.g., with unequal spacing about the perimeter of the reflector 1004 or otherwise in a non-symmetrical fashion such that a component attached to the mounts 1012 may only be secured in a single orientation. The polarizer 904 couples to the mounts 1012 and therefore the non-symmetrical arrangement of the mounts 1012 ensures that the polarizer is mounted in a correct orientation such that the polarization of the light passing through the polarizer 904 is such that the light will traverse the beam splitter 810.

FIG. 11A illustrates a side view of the light management system 1000 of FIG. 10, in accordance with one or more examples. FIG. 11B illustrates a detail view of a portion of the light management system 1000 of FIG. 11A, in accordance with one or more examples. The light management system 1000 as depicted in FIGS. 11A and 11B includes the emitter board 1002, reflector 1004, LED 1006, recess 1008, and other such components as shown and described herein.

The detail view shown in FIG. 11B illustrates an LED 1006 situated within a recess 1008 of the reflector 1004. The recesses 1008 are shown with a shape that forms a parabolic curve in the section view shown in FIGS. 11A and 11B. The parabolic curve provides that light emitted by the LEDs 1006 is reflected into columnar light parallel with a light axis 1102. Accordingly, the light from each of the LEDs 1006 is directed parallel to the light axis 1102 towards a patient. In some examples, based on the array of the LEDs 1006, the recesses 1008 may intersect one another. In some examples, the spacing between the LEDs 1006 may be sufficient for the recesses 1008 to extend with the parabolic curvature from the LED 1006 to the output surface of the reflector 1004. The recesses 1008 may contact and/or be adjacent to the surface of the dome of the LED 1006 at a point 1106. Light emitted from the LED 1006 on a first side of the point 1106 may be reflected by the reflector 1004. Light emitted from the LED 1006 on a second side of the point 1106 is reflected by an undercut 1108 that prevents stray light from escaping from the reflector 1004 and also provides clearance for a base of the LED 1006 where the LED 1006 is connected to the emitter board 1002. The undercut 1108 may be shaped as a bevel and therefore form a beveled undercut to trap light emitted at or near the base of the LED 1006.

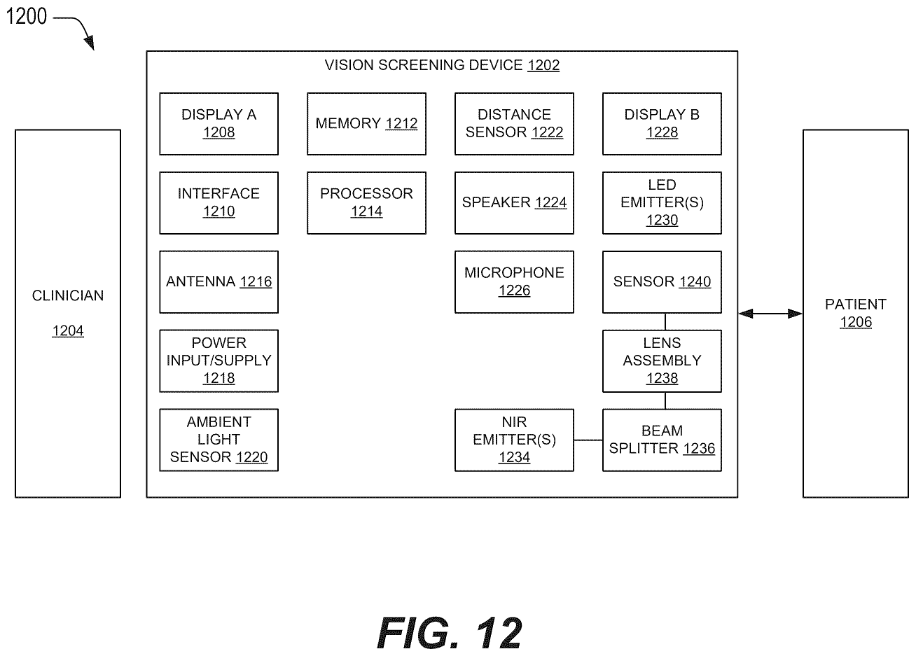

FIG. 12 illustrates a system architecture 1200 of a vision screening device 1202, in accordance with one or more examples. The vision screening device 1202 may be used by a clinician 1204 interacting with a patient 1206, as described herein. The system architecture 1200 illustrates a subset of components that may be included as part of the vision screening device 1202 in an illustrative example. The vision screening device 1202 may include additional components not shown herein that may be used to perform the operations described herein and/or support one or more additional components. Further, other components may be substituted for one or more of the shown components that may perform the same or similar functions.

The vision screening device 1202 includes a display A 1208 that is clinician-facing and provides an interface for a clinician to input data as well as receive outputs from the vision screening device. A further interface 1210, which may include a touchscreen display, provides the clinician with an ability to interact with the vision screening device, for example to input demographic data, patient data, select types of exams to perform, and other such interactions. A memory 1212, such as a non-transitory computer-readable medium may include specific instructions stored thereon (e.g., software) that, when executed by a processor 1214, cause the processor to perform various operations related to vision screening and/or other operations.

The vision screening device 1202 further includes an antenna 1216 that may provide a wireless communication with one or more other devices or systems such as a system that stores or provides access to electronic medical records for patients. A power input/supply 1218 provides for portable power (e.g., batteries) as well as a system for charging batteries and/or providing consistent power to one or more other components of the vision screening device 1202.

An ambient light sensor 1220 may be used to detect light levels and/or characteristics of ambient light in the environment surrounding the visions screening device. The ambient light sensor 1220 may detect a brightness of the environment and may also detect illumination colors, for instance if the lighting in a particular area is “warm” or “cool” relating to the temperature of the lighting. In some examples, the ambient light sensor 1220 may be used to detect potential environmental light that may interfere with the vision screening tests, such as a prevalence of NIR light from a nearby source. Such information may be displayed on display A 1208 for the clinician to either acknowledge or work to adjust environmental conditions to be more conducive to particular eye exams.

A distance sensor 1222 may be used to determine a distance between the vision screening device 1202 and the patient 1206. The distance sensor 1222 may include a range finding sensor of any suitable type for detecting a distance between the location of the sensor (at the vision screening device 1202) and the patient 1206. In examples, the distance sensor 1222 may be positioned behind a patient-facing cover on a side of the vision screening device 1202 opposite display A 1208. Accordingly, as with the imaging components, the distance sensor 1222 may enable the housing of the vision screening device 1202 to remain compact and user-friendly by presenting a slim and easily held device that is also portable.

A speaker 1224 may provide audible outputs or cues, such as a beeping sound indicative of the patient being at or near the predetermined distance. In an example, an audible cue may increase in tone and/or frequency of a repeated sound as the patient 1206 approaches the predetermined distance. A microphone 1226 may be used to gather audible data, for example to enable the vision screening device 1202 to recognize the patient reading a particular chart and to provide evaluation of their performance reading the symbols displayed on display B 1228, that is user-facing, as described herein.

LED emitter(s) 1230 may include NIR emitters 1234 as well as white light emitters that provide light to reflect off the patient 1206, with the reflected radiation received through a beam splitter 1236 and lens assembly 1238 before arriving at the sensor 1240, as shown and described with respect to FIGS. 6-11B herein.

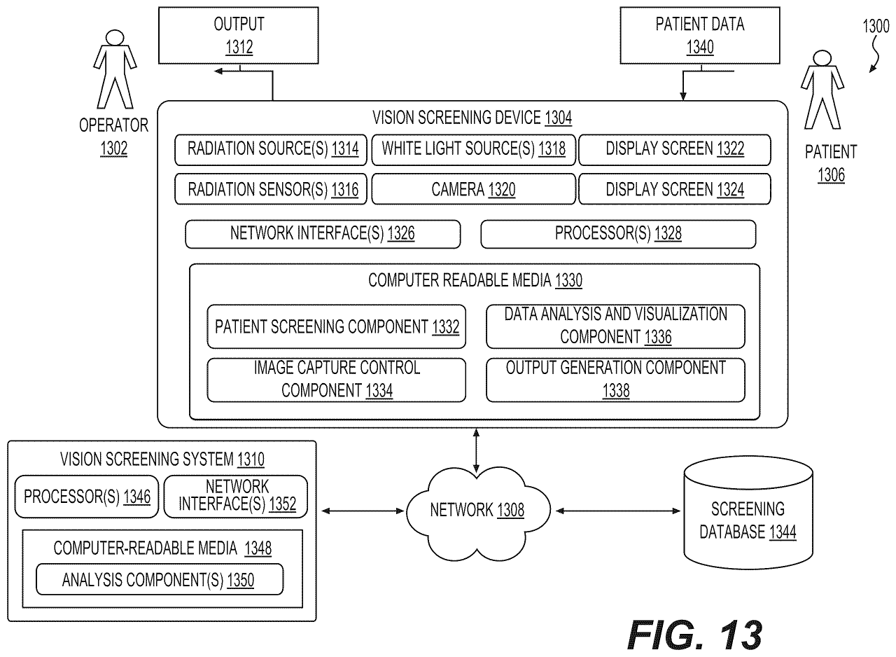

FIG. 13 illustrates a system architecture of a vision screening system for performing vision screening tests and recording test result data for patients, in accordance with one or more examples. As illustrated in FIG. 13, in some examples an operator 1302 may administer vision screening tests, via a vision screening device 1304, on a patient 1306 to determine eye health of the patient 1306. As described herein, the vision screening device 1304 may perform one or more vision screening tests, including screening for diseases and/or abnormalities of eye(s) when the eyes are illuminated by visible light. In addition, the vision screening device 1304 may also be configured to perform other vision screening tests, such as a visual acuity test, a refractive error test, an accommodation test, dynamic eye tracking tests, color vision screening test, visible light test, and/or any other vision screening tests, configured to evaluate and/or diagnose the vision health of the patient 1306. In examples, the vision screening device 1304 may comprise a portable device configured to perform the one or more vision screening tests. Due to its portable nature, the vision screening device 1304 may perform the vision screening tests at any location, from conventional screening environments, such as schools and medical clinics, to physician's offices, hospitals, eye care facilities, and/or other remote and/or mobile locations. Furthermore, the vision screening tests may be conducted at a common distance, providing for a more efficient process and resulting in the vision screening device 1304 being capable of being used for large group testing. It is also envisioned that the vision screening device 1304 may be used for administering vision screening tests to all age groups, including newborns and young children and geriatric patients.

As described herein, the vision screening device 1304 may be configured to perform one or more vision screening tests on the patient 1306. In examples, one or more vision screening tests may include illuminating the eye(s) of the patient 1306 with infrared or near-infrared (NIR) radiation and capturing reflected radiation from the eye(s) of the patient 1306. For example, U.S. Pat. No. 13,237,846, the entire disclosure of which is incorporated herein by reference, describes systems and methods for determining refractive error based on photorefraction using pupil images captured under different illumination patterns generated by near-infrared (NIR) radiation sources. In other examples, vision screening tests, such as the red reflex test, may include illuminating the eye(s) of the patient 1306 with visible light, and capturing color image(s) of the eye(s) under visible light illumination. The vision screening device 1304 may acquire data comprising color images and/or video data of the eye(s) under visible light illumination, and detect pupils, retinas, and/or lenses of the eye(s) of the patient 1306. This data may be used to determine differences between left and right eyes, compare the captured images with standard images, or generate visualizations to assist the operator 1302 or a clinician in diagnosing diseases and abnormalities of the eye(s) of the patient. The vision screening device 1304 may transmit the data, via a network 1308, to a vision screening system 1310 for analysis to determine an output 1312 associated with the patient 1306. Alternatively, or in addition, the vision screening device 1304 may perform some or all of the analysis locally to determine the output 1312. Indeed, in any of the examples described herein, some or all of the disclosed methods may be performed in whole or in part by the vision screening device 1304 independently (e.g., without the vision screening system 1310 or its components), or by the vision screening system 1310 independently (e.g., without the vision screening device 1304 or its components). For instance, in some examples, the vision screening device 1304 may be configured to perform any of the vision screening tests, and/or other methods described herein without being connected to, or otherwise in communication with, the vision screening system 1310 via the network 1308. In other example, the vision screening system 1310 may include one or more components that are similar to and/or the same as those included in the vision screening device 1304, and thus, the vision screening system 1310 may be configured to perform any of the vision screening tests, and/or other methods described herein without being connected to, or otherwise in communication with, the vision screening device 1304.

As shown schematically in FIG. 13, the vision screening device 1304 may include one or more radiation source(s) 1314 configured to perform functions associated with administering one or more vision screening tests. The radiation source(s) 1314 may comprise individual radiation emitters, such as light-emitting diodes (LEDs), which may be arranged in a pattern to form an LED array. In examples, the radiation source(s) 1314 may include near-infrared (NIR) radiation emitters, such as NIR LEDs, for measuring the refractive error of the eye(s) of the patient 1306 using photorefraction methods. The NIR radiation emitters of the radiation source(s) 1314 may also be used for measuring the gaze angle or gaze direction of the eye(s) of the patient 1306. In addition, the radiation source(s) 1314 may also include color LEDs for generating color stimuli for display to the patient 1306 during a color vision screening test.

The vision screening device 1304 may also include one or more radiation sensor(s) 1316, such as infrared cameras, configured to capture reflected radiation from the eye(s) of the patient during the vision screening test(s). For example, the vision screening device 1304 may emit, via the radiation source(s) 1314, one or more beams of radiation, and may be configured to direct such beams at the eye(s) of the patient 1306. The vision screening device 1304 may then capture, via the radiation sensor(s) 1316, corresponding radiation that is reflected back (e.g., from pupils of the eye(s)). In examples, the radiation sensor(s) 1316 may comprise NIR radiation sensor(s) to capture reflected NIR radiation while the eye(s) of the patient 1306 are illuminated by the radiation source(s) 1314. The data captured by the radiation sensor(s) 1316 may be used in the measurement of the refractive error and/or gaze angle(s) of the eye(s) of the patient 1306. The data may include images and/or video of the pupils, retinas, and/or lenses of the eyes of the patient 1306. In some examples, the images and/or video may be in grayscale. The data may be captured intermittently, during specific periods of the vision screening test(s), or during the entire duration of the test(s). Additionally, the vision screening device 1304 may process the image(s) and/or video data to determine change(s) in the refractive error and/or gaze angle(s) of the eye(s) of the patient 1306. The grayscale images of the eye(s) captured under NIR illumination may also be used for screening for diseases and abnormalities of the eye(s) such as ametropia, strabismus, and occlusions. As described herein, in some examples, the radiation sensor(s) 1316 may be combined with camera 1320 into a single sensing component that detects both visible light and NIR.

In examples, the vision screening device 1304 may further include visible white light source(s) 1318 and camera 1320 configured to capture color images and/or video of the eyes under illumination by the white light source(s) 1318. The white light source(s) 1318 may comprise light-emitting diodes (LEDs) such as an array of LEDs configured to produce white light e.g., a blue LED with a phosphor coating to convert blue light to white light, or a combination of red, blue, and green LEDs configured to produce white light by varying intensities of individual red, blue and green LED activation. Individual LEDs of the array of LEDs may be arranged in a pattern configured to be individually operable to provide illumination from different angles during the vision screening test(s). The white light source(s) 1318 may also be configured to produce white light of different intensity levels. The camera 1320 may be configured to capture white light reflected from the eyes of the patient to produce digital color images and/or video. The camera 1320 may comprise a high-resolution, auto-focus digital camera with custom optics for imaging eyes in clinical applications. The color images and/or video captured by the camera 1320 may be stored in various formats, such as JPEG, BITMAP, TIFF, etc. (for images) and MP4, MOV, WMV, AVI etc. (for video). In some examples, pixel values in the color images and/or video may be in a RGB (red, green, blue) color space. The color images and/or video of the eye(s) captured under white light illumination may be used for screening for diseases and abnormalities of the eye(s) such as cataracts, media opacities in aqueous and vitreous humors, tumors, retinal cancers and detachment, and the like. In addition, the color images and/or video may be used in conjunction with the grayscale images captured under NIR illumination to generate visualizations to assist in the detection of a wide range of disease conditions of the eye(s).

The vision screening device 1304 may also include one or more display screen(s), such as display screen 1322 and display screen 1324, which may be color LCD (liquid crystal display), or OLED (organic light-emitting diode) display screens. The display screen 1322 may be an operator display screen facing a direction towards the operator 1302, configured to provide information related to the vision screening tests to the operator 1302. In any of the examples described herein, the display screen 1322 facing the operator 1302 may be configured to display and/or otherwise provide the output 1312 generated by the vision screening device 1304 and/or generated by the vision screening system 1310. The output 1312 may include testing parameters, current status and progress of the screening test(s), measurements(s) determined during the test(s), image(s) captured or generated during the screening test(s), a diagnosis determined based on one or more tests, and/or a recommendation associated with the diagnosis. The display screen 1322 facing the operator 1302 may also display information related to or unique to the patient, and the patient's medical history.

In some examples, the vision screening device 1304 may also include a display screen 1324 facing in a direction towards the patient 1306 and configured to display content to the patient 1306. The content may include attention-attracting images and/or video to attract attention of the patient and hold the patient's gaze towards the vision screening device 1304. Content corresponding to various vision screening test(s) may also be presented to the patient 1306 on the display screen 1324. For example, the display screen 1324 may display color stimuli to the patient 1306 during a color vision screening test, or a Snellen eye chart during a visual acuity screening test. The display screen 1322 and display screen 1324 may be integrated with the vision screening device 1304, or may be external to the device, and under computer program control of the vision screening device 1304.

The vision screening device 1304 may transmit the data captured by the radiation sensor(s) 1316 and/or the camera 1320, via the network 1308, using network interface(s) 1326 of the vision screening device 1304. In addition, the vision screening device 1304 may also similarly transmit other testing data associated with the vision screening test(s) being administered, (e.g., type of test, duration of test, patient identification and the like). The network interface(s) 1326 of the vision screening device 1304 may be operably connected to one or more processor(s) 1328 of the vision screening device 1304, and may enable wired and/or wireless communications between the vision screening device 1304 and one or more components of the vision screening system 1310, as well as with one or more other remote systems and/or other networked devices. For instance, the network interface(s) 1326 may include a personal area network component to enable communications over one or more short-range wireless communication channels, and/or a wide area network component to enable communication over a wide area network. In any of the examples described herein, the network interface(s) 1326 may enable communication between, for example, the processor(s) 1328 of the vision screening device 1304, and the vision screening system 1310, via the network 1308. The network 1308 shown in FIG. 13 may be any type of wireless network or other communication network known in the art. Examples of network 1308 include the Internet, an intranet, a wide area network (WAN), a local area network (LAN), and a virtual private network (VPN), cellular network connections and connections made using protocols such as 802.11a, b, g, n and/or ac.

The vision screening system 1310 may be configured to receive data, from the vision screening device 1304 and via the network 1308, collected during the administration of the vision screening test(s). In some examples, based at least in part on processing the data, the vision screening system 1310 may determine the output 1312 associated with the patient 1306. For example, the output 1312 may include a recommendation and/or diagnosis associated with eye health of the patient 1306, based on an analysis of the color image data and/or NIR image data indicative of diseases and/or abnormalities associated with the eye(s) of the patient 1306. The vision screening system 1310 may communicate the output 1312 to the processor(s) 1328 of the vision screening device 1304 via the network 1308. As noted above, in any of the examples described herein one or more such recommendations, diagnoses, or other outputs may be generated, alternatively or additionally, by the vision screening device 1304.

As described herein, a processor, such as the processor(s) 1328, can be a single processing unit or a number of processing units, and can include single or multiple computing units or multiple processing cores. The processor(s) 1328 can be implemented as one or more microprocessors, microcomputers, microcontrollers, digital signal processors, central processing units, state machines, logic circuitries, and/or any devices that manipulate signals based on operational instructions. For example, the processor(s) 1328 can be one or more hardware processors and/or logic circuits of any suitable type specifically programmed or configured to execute the algorithms and processes described herein. As shown schematically in FIG. 13, the vision screening device 1304 may also include computer-readable media 1330 operably connected to the processor(s) 1328. The processor(s) 1328 can be configured to fetch and execute computer-readable instructions stored in the computer-readable media 1330, which can program the processor(s) 1328 to perform the functions described herein.

The computer-readable media 1330 may include volatile and nonvolatile memory and/or removable and non-removable media implemented in any type of technology for storage of information, such as computer-readable instructions, data structures, program modules, or other data. Such computer-readable media 1330 can include, but is not limited to, RAM, ROM, EEPROM, flash memory or other memory technology, optical storage, solid state storage, magnetic tape, magnetic disk storage, RAID storage systems, storage arrays, network attached storage, storage area networks, cloud storage, or any other medium that can be used to store the desired information and that can be accessed by a computing device. The computer-readable media 1330 can be a type of computer-readable storage media and/or can be a tangible non-transitory media to the extent that when mentioned, non-transitory computer-readable media exclude media such as energy, carrier signals, electromagnetic waves, and signals per se.

The computer-readable media 1330 can be used to store any number of functional components that are executable by the processor(s) 1328. In examples, these functional components comprise instructions or programs that are executable by the processor(s) 1328 and that, when executed, specifically configure the one or more processor(s) 1328 to perform actions associated with one or more of the vision screening tests used for the detection and diagnosis of diseases and abnormalities of the eye(s). For example, the computer-readable media 1330 may store one or more functional components for administering vision screening tests, such as a patient screening component 1332, an image capture control component 1334, a data analysis and visualization component 1336, and/or an output generation component 1338, as illustrated in FIG. 13. At least some of the functional components of the vision screening device 1304 will be described in detail below.

In examples, the patient screening component 1332 may be configured to store and/or access patient data 1340 associated with the patient 1306. For example, the patient data 1340 may include demographic information such as name, age, ethnicity, and the like. When the vision screening device 1304 and/or vision screening system 1310 initiates a vision screening test, the patient 1306 may provide, or the operator 1302 may request, from the patient 1306 or a guardian of the patient 1306 the patient data 1340 regarding the patient's demographic information, medical information, preferences, and the like. In such examples, the operator 1302 may request the data while the screening is in progress, or before the screening has begun. In some examples, the operator 1302 may be provided with predetermined categories associated with the patient 1306, such as predetermined age ranges (e.g., newborn to six months, six to twelve months, one to five years old, etc.), and may request the patient data 1340 in order to select the appropriate category associated with the patient 1306. In other examples, the operator 1302 may be provided a free form input associated with the patient data 1340. In still further examples, an input element may be provided to the patient 1306 directly.

The vision screening device 1304 may use a sensor such as a distance sensor to detect distance data corresponding to a distance between the vision screening device 1304 and the patient 1306. The distance may be used to position the vision screening device 1304 relative to the patient, for example to reach a particular spacing or distance between the patient 1306 and the vision screening device 1304 such as described herein. Additional sensors such as ambient light sensors may be used for determining environmental conditions, such as whether environmental lighting conditions are suitable for a particular eye exam to take place.

Alternatively, or in addition, the vision screening device 1304 and/or vision screening system 1310 may determine and/or detect the patient data 1340 during the vision screening test. For example, the vision screening device 1304 may include one or more digital cameras, motion sensors, proximity sensors, or other image capture devices configured to collect images and/or video data of the patient 1306, and one or more processors of the vision screening device 1304 may analyze the data to determine the patient data 1340, such as the age category of the patient 1306 or a distance of the patient 1306 from the screening device. For example, the vision screening device 1304 may be equipped with a range finder, such as an ultra-sonic range finder, an infrared range finder, and/or any other proximity sensor that may be able to determine the distance of the patient 1306 from the screening device.

Alternatively, or in addition, the vision screening device 1304 may be configured to transmit the images/video data to the vision screening system 1310, via the network 1308, for analysis to determine the patient data 1340. Further, the patient screening component 1332 may be configured to receive, access, and/or store the patient data 1340 associated with the patient 1306 and/or additional patients. For example, the patient screening component 1332 may store previous patient information associated with the patient 1306 and/or other patients. For instance, the patient screening component 1332 may store previous screening history of the patient 1306, including data from previous screening such as color images, NIR images, and/or video of the eye(s) of the patient 1306. The patient screening component 1332 may receive the patient data 1340 and/or may access such information via the network 1308. For example, the patient screening component 1332 may access an external database, such as screening database 1344, storing data associated with the patient 1306 and/or other patients. The screening database 84 may be configured to store the patient data 1340 in association with a patient ID. When the operator 1302 and/or the patient 1306 enters the patient ID, the patient screening component 1332 may access or receive the patient data 1340 stored in association with the patient ID of the patient 1306.

In examples, the patient screening component 1332 may be configured to determine the vision screening test(s) to administer to the patient 1306 based at least in part on the patient data 1340. For example, the patient screening component 1332 may utilize the patient data 1340 to determine a testing category that the patient 1306 belongs to (e.g., a testing category based on age, medical history, etc.). The patient screening component 1332 may determine the vision screening test(s) to administer based on the testing category. For example, if the patient data 1340 indicates that the patient is a newborn, the selected vision screening test(s) may include screening for congenital conditions of the eye such as congenital cataracts, retinoblastoma, opacities of the cornea, strabismus and the like. In addition, eye abnormalities may be associated with systemic inherited diseases such as Marfan syndrome and Tay-Sachs disease. For example, a screening test for a characteristic red spot in the eye may indicate Tay-Sachs disease. As another example, if the patient data 1340 indicates that the patient is above fifty years old, the patient screening component 1332 may determine that the vision screening test(s) include screening for onset of cataracts, macular degeneration and other age-related eye diseases.

The patient screening component 1332 may also determine vision screening test(s) based on the patient's medical history. For example, the screening database 1344 may store, in the patient data 1340, medical history associated with previous vision screening tests of the patient 1306, including test results, images of the eye(s), measurements, recommendations, and the like. The patient screening component 1332 may access the patient data 1340 including medical history from the screening database 1344 and determine vision screening test(s) to administer to monitor status and changes in previously detected vision health issues. For example, if a progressive eye disease, such as onset of cataracts or macular degeneration, was detected in a previous screening, further screening may be administered to track the development of the disease. As another example, if the patient 1306 had surgery for removal of a tumor of the eye(s), the vision screening test(s) may include screening for further tumors or scarring in the eye(s).

In some examples, the computer-readable media 1330 may additionally store an image capture control component 1334. The image capture control component 1334 may be configured to operate the radiation source(s) 1314, the radiation sensor(s) 1316, the white light source(s) 1318, and the camera 1320 of the vision screening device 1304, so that images of the eye(s) are captured under specific illumination conditions required for each particular vision screening test(s). As discussed, the radiation source(s) 1314 may include NIR LEDs for illuminating the eye(s) during capture of grayscale images for measuring the refractive error and/or gaze angle of the eye(s) of the patient 1306, and the white light source(s) 1318 may include white light LEDs for illuminating the eye(s) during capture of color images of the eye(s) by the camera 1320. In examples, the image capture control component 1334 may generate commands to operate and control the individual radiation sources, such as LEDs of the NIR LEDs, as well as the LEDs of the white light source(s) 1318. Control parameters of the LEDs may include intensity, duration, pattern and cycle time. For example, the commands may selectively activate and deactivate the individual LEDs of the radiation source(s) 1314 and white light source(s) 1318 to produce illumination from different angles as needed by the vision screening test(s) indicated by the patient screening component 1332. The image capture control component 1334 may activate the NIR LEDs of the radiation source(s) 1314 used for measuring the refractive error and/or gaze angle of the eye(s) of the patient 1306 in synchronization with the capture of images of the eye(s) by the radiation sensor(s) 1316 during the performance of a vision screening test. Similarly, the image capture control component 1334 may activate the LEDs of the white light source(s) 1318 in synchronization with the capture of color images of the eye(s) by the camera 1320.

The individual radiation sources, such as LEDs, of the radiation source(s) 1314 or the white light source(s) 1318 may be controlled by the image capture control component 1334 according to control parameters stored in the computer-readable media 1330. For instance, control parameters may include intensity, duration, pattern, cycle time, and so forth, of the NIR LEDs of the radiation source(s) 1314 and/or the LEDs producing white light of the white light source(s) 1318. For example, the image capture control component 1334 may use the control parameters to determine a duration that individual LEDs of the radiation source(s) 1314 and white light source(s) 1318 emit radiation (e.g., 50 milliseconds, 800 milliseconds, 200 milliseconds, etc.). Additionally, the image capture control component 1334 may utilize the control parameters to alter an intensity and display pattern of NIR LEDs of the radiation source(s) 1314 for the determination of refractive error of the eye(s) based on photorefraction and/or gaze angle of the eye(s). With respect to intensity, the image capture control component 1334 may control parameters to direct the LEDs of the white light source(s) 1318 to emit light at an intensity that is bright enough to capture a color image of the eye(s) using the camera 1320, while also limiting brightness to avoid or reduce pupil constriction or accommodation. The image capture control component 1334 may also control the intensity of the white light source(s) 1318 to gradually increase the intensity at a certain rate while activating the camera 1320 to capture images and/or video of the eyes to record response of the pupils of the patient's eyes to the increasing intensity of illumination.

Further, the image capture control component 1334 may order the emission of radiation from the radiation source(s) 1314 and white light source(s) 1318 so that the NIR LEDs are activated and the images of the eye(s) under NIR radiation are captured before the activation of the LEDs of the white light source(s) 1318. In some examples, this ordering may prevent the constriction of the pupils of the eye(s) in response to white light impinging upon them, and/or may allow for the capture of images of the internal structures of the eye(s) without the need for dilating the pupils of the patient 1306. In some examples, the image capture control component 1334 may additionally control the radiation source(s) 1314 and white light source(s) 1318 to generate patterns such as circular patterns, alternating light patterns, flashing patterns, patterns of shapes such as circles or rectangles, and the like to attract the attention of the patient 1306, and/or control color LEDs of the radiation source(s) 1314 and white light source(s) 1318 to display color stimuli such as color dot patterns to the patient 1306 during vision screening.

The image capture control component 1334 may also control the radiation sensor(s) 1316 and the camera 1320 to capture images and/or video of the eye(s) of the patient 1306 during the administration of the vision screening test(s). For example, the radiation sensor(s) 1316 may capture data indicative of reflected radiation from the eye(s) of the patient 1306 during the activation of one or more of the radiation source(s) 1314. The data may include grayscale image data and/or video data of the eye(s). The image capture control component 1334 may synchronize the camera 1320 to capture color image(s) and/or video data of the eye(s) with the activation of the white light source(s) 1318 so that the eye(s) are illuminated by white light radiation during the capture of the color image and/or video data. In some examples, images of the left and the right eye may be captured under different illumination conditions (e.g., from a different individual source), so that the relative angle of illumination with the optical axis of the particular eye is the same for the left and the right eye. In other examples, images of both eyes may be captured simultaneously under the same illumination. As described herein, the image capture control component 1334 of the vision screening device 1304 may generate grayscale images of the eye(s) illuminated under NIR radiation, and color images of the eye(s) illuminated under white light. Capturing both the grayscale images and the color images may enable the detection of a wider range of diseases and abnormalities of the eyes.

In some examples, the computer-readable media 1330 may also store a data analysis and visualization component 1336. The data analysis and visualization component 1336 may be configured to analyze the image and/or video data collected, detected, and/or otherwise captured by components of the vision screening device 1304 (e.g., by the radiation sensor(s) 1316, and the camera 1320) during one or more vision screening tests. For example, the data analysis and visualization component 1336 may analyze the data to determine location of the pupils of the eye(s) in the images, and identify a portion of the image(s) corresponding to the pupil (e.g., pupil image(s)). The data analysis and visualization component 1336 may analyze the pupil image(s) to determine characterizations of appearance of the pupil(s) in the pupil image(s). For example, in the instance of the color image(s) captured by the camera 1320, the characterizations may include an average color value, variance of color values, measure of uniformity, presence of inclusions, and the like. In the instance infrared image(s) captured by the radiation sensor(s) 1316, the characterizations may include average grayscale value and variance of grayscale values, instead of the color, in addition to measures of uniformity and the presence of inclusions. The data analysis and visualization component 1336 may further compare the left pupil image(s) and the right pupil image(s) to determine differences in appearance between the left and right pupils. For example, the differences may correspond to a difference in average color value or average grayscale value between the left pupil image(s) and right pupil image(s). The data analysis and visualization component 1336 may also compare the pupil image(s) with standard pupil image(s) and/or pupil image(s) of the patient 1306 captured during previous vision screening(s) to determine differences in appearance, such as differences in average color value or grayscale value, differences in the measure of uniformity, differences in detected inclusions, and the like. In any of the examples above, all captured image(s) or a subset of the captured grayscale and/or color images may be used to determine differences. In some examples, grayscale image(s) may not be used, and the difference may be determined based on the color image(s). It is to be noted that pixels of grayscale images may also be considered to have a color value, wherein the color value is determined by using the same grayscale value for each of the three-color channels (e.g., RGB). For example, a pixel with a grayscale value of 828, may be determined to have a color value of (128, 828, 828) in the RGB color space. The data analysis and visualization component 1336 may also apply additional image processing steps to the grayscale image(s) and/or the color image(s) which may improve detection of disease states. For example, images may be sharpened, specific colors may be boosted or attenuated, color or brightness of the images may be balanced, and the like.

Further, the data analysis and visualization component 1336 may be configured to receive, access, and/or analyze standard data associated with vision screening. For example, the data analysis and visualization component 1336 may be configured to access or receive data from one or more additional databases (e.g., the screening database 1344, a third-party database, etc.) storing testing data, measurements, and/or values indicating various thresholds or ranges within which measured values should lie. Such thresholds or ranges may be associated with patients having normal vision health and may be learned or otherwise determined from standard testing. The data analysis component and visualization component 1336 may utilize the standard data for comparison with the average values and differences determined during the vision screening test(s) as described above. For example, the standard data may indicate a threshold or a range for a difference between color values of the left and right pupil images, where a difference greater than the threshold, or outside the range, corresponds to an abnormality in the eye(s) of the patient. Alternatively, or in addition, the data analysis and visualization component 1336 may access a previous vision screening of the patient 1306 and compare the values and differences with corresponding data from the previous screening(s). For example, an average color value of the pupil may be compared with an average color value from a previous screening to determine a difference. This difference may then be compared with standard thresholds or ranges to determine presence of an abnormality, as described above. Separate threshold(s) and/or range(s) may be indicated in the standard data for different types of diseases and abnormalities. In addition, the threshold(s) and/or range(s) associated with the vision screening test may also be based on the testing category of the patient 1306 (e.g., the age group or medical history of the patient 1306), where the threshold(s) and/or range(s) may be different for different testing categories. The data analysis and visualization component 1336 may store as a part of the patient data 1340, images and/or video captured or generated during the vision screening test(s), measurements associated with the vision screening test(s), test results, and other data in a database(e.g., in the screening database 1344) for comparison of data over time to monitor vision health status and changes in vision health. In some examples, the stored images may include images of the face or partial face (e.g., eyes and part of nose) of the patient 1306.

Based on the comparison with a threshold and/or range described above, the data analysis and visualization component 1336 may generate a normal/abnormal or a pass/refer determination for each of the eyes of the patient 1306. For example, if all values and differences measured are less than on equal to corresponding threshold(s), or fall within the corresponding range(s) of the standard data, a “normal” or “pass” determination may be made by the data analysis and visualization component 1336, and an “abnormal” or “refer” determination made otherwise to indicate a referral for further screening. Alternatively, or in addition, the data analysis and visualization component 1336 may generate a normal/abnormal determination for each of the diseases and/or abnormalities screened for during the vision screening session.

In examples, the data analysis and visualization component 1336 may utilize one or more machine learning techniques to generate a diagnosis of specific diseases and/or types of abnormalities. For example, machine learning (ML) models may be trained with normal images of eyes, and images of eyes labeled as exhibiting various disease conditions and abnormalities. The trained ML model(s) may then generate an output indicating a disease or abnormality diagnosis when provided, as input, an image of the eye captured during the vision screening of the patient 1306. In such examples, the data analysis and visualization component 1336 may directly generate the output by providing an image of the eye as input to the trained ML model(s), without computing differences between pupil images or applying comparisons with a threshold and/or range. In some examples, a plurality of trained ML model(s) may be used, each ML model being trained to detect a specific disease or abnormality. In such examples, each ML model outputs a binary present/absent indication to indicate if the input image exhibits the disease or abnormality that the ML model is trained to detect. The data analysis and visualization component 1336 may provide an image of the eye as input to each ML model of the plurality of trained ML model(s) for detecting one or more of a plurality of diseases and abnormalities. In examples, the ML models may be neural networks, including convolutional neural networks (CNNs). In other examples, the ML models can also include regression algorithms, decision tree algorithms, Bayesian classification algorithms, clustering algorithms, support vector machines (SVMs) and the like.

The data analysis and visualization component 1336 may also generate visualizations of the eye(s) using the image(s) and/or video data captured by the radiation sensor(s) 1316 and/or the camera 1320. For example, a first visualization may include a composite image of the eye(s) incorporating both color information from the color image(s) and grayscale information from the grayscale image(s) captured by the radiation sensor(s) 1316 under NIR illumination. The generation of the first visualization may include detection and identification of structures of the eye(s) such as pupils and/or lenses, followed by registration of the grayscale image(s) and the color image(s) so that the pupils are located in the same position in both types of image(s). The composite image may then be generated by using grayscale pixel values from the grayscale image(s) in some portions of the composite image and color pixel values from the color image(s) in other portions of the composite image. The portions of the composite image using grayscale pixel values and the portions using color pixel values may correspond to areas depicting different structures of the eye(s) (e.g., fovea, retina, cornea etc.). The composite image may more clearly delineate structures of the eye(s) for improved detection and assessment of diseases and/or abnormalities of the eye(s).

In another example, a second visualization may include a sequence of still images, or an animated video comprising the sequence of still images. In some instances, the sequence of images may be captured by the radiation sensor(s) 1316 or the camera 1320 while the eye(s) are illuminated by the radiation source(s) 1314 or white light source(s) 1318 at a progression of different angles along different axes with respect to the optical axis. In some examples, the visualization may include graphics and/or color-coding indicative of areas of the image of the eye(s) that are flagged as being abnormal. As described herein, the data analysis and visualization component 1336 of the vision screening device 1304 may process the grayscale images and the color images of the eye(s) captured during the administration of the vision screening test(s), to determine diseases and/or abnormalities associated with the eye(s) of the patient. In addition, the data analysis and visualization component 1336 may generate, based on the grayscale and color images, visualizations of the eye(s) that aid a clinician or an operator of the vision screening device 1304 to identify diseases and/or abnormalities of the eye(s).

The computer-readable media 1330 may additionally store an output generation component 1338. The output generation component 1338 may be configured to receive, access, and/or analyze data from the data analysis and visualization component 1336, and generate the output 1312. For example, the output generation component 1338 may utilize the normal/abnormal determinations of the data analysis and visualization component 1336 to generate a recommendation in the output 1312. The recommendation may indicate whether the screening results of the patient 1306 indicate normal eye health, or further screening is needed based on one or more of the screening tests resulting in an “abnormal” finding. In addition, the output generation component 1338 may incorporate all or a subset of the visualizations generated by the data analysis and visualization component 1336 into the output 1312 for aiding in diagnosis of the condition of the eye(s). Portions of the images and/or video captured by the radiation sensor(s) 1316 or the camera 1320 may also be included in the output 1312. Additionally, if abnormality is determined, the output generation component 1338 may incorporate a likely diagnosis into the output 1312 based on the analysis by the data analysis and visualization component 1336. The output 1312 may be presented to the operator of the device via an interface of the device (e.g., on the display screen 1322 of the vision screening device 1304). In examples, the operator display screen may not be visible to the patient, e.g., the operator display screen may be facing in a direction opposite the patient. The output generation component 1338 may also store the output 1312, which may include a recommendation, diagnosis, measurements, captured images/video and/or the generated visualizations in a database, such as the screening database 1344, for evaluation by a clinician, or for access during subsequent vision screening(s) of the patient 1306. The screening database 1344 may provide access to authorized medical professionals to enable printing of reports or further assessment of the data related to the screening of the patient 1306.

Although FIG. 13 illustrates example processor(s) 1328 and computer-readable media 1330 storing a patient screening component 1332, an image capture control component 1334, a data analysis and visualization component 1336, an output generation component 1338 and/or other components and/or other items as components of the vision screening device 1304, in any of the examples described herein, the vision screening system 1310 may include similar components and/or the same components. In such examples, the vision screening system 1310 may include processor(s) 1346 and computer-readable memory 1348 that are configured to perform the functions of some or all of the components in the computer-readable media 1330 of the vision screening device 1304. For example, one or more of the components of the computer-readable media 1330 may be included in analysis component(s) 1350 of computer-readable memory 1348 and be executable by the processor(s) 1346. In such examples, the vision screening system 1310 may communicate with the vision screening device 1304 using network interface(s) 1352, and via the network 1308, to receive data from the vision screening device 1304 and send results (e.g., output 1312), back to the vision screening device 1304. The vision screening system 1310 may be implemented on a computer proximate the vision screening device 1304 or may be at a remote location. For example, the vision screening system 1310 may be implemented as a cloud service on a remote cloud server.

The network interface(s) 1352 may enable wired and/or wireless communications between the components and/or devices shown in system 1300 and/or with one or more other remote systems, as well as other networked devices. For instance, at least some of the network interface(s) 1352 may include a personal area network component to enable communications over one or more short-range wireless communication channels. Furthermore, at least some of the network interface(s) 1352 may include a wide area network component to enable communication over a wide area network. Such network interface(s) 1352 may enable, for example, communication between the vision screening system 1310 and the vision screening device 1304 and/or other components of the system 1300, via the network 1308. For instance, the network interface(s) 1352 may be configured to connect to external databases (e.g., the screening database 1344) to receive, access, and/or send screening data using wireless connections. Wireless connections can include cellular network connections and connections made using protocols such as 802.11a, b, g, and/or ac. In other examples, a wireless connection can be accomplished directly between the vision screening device 1304 and an external system using one or more wireless protocols, such as Bluetooth, Wi-Fi Direct, radio-frequency identification (RFID), infrared signals, and/or Zigbee. Other configurations are possible. The communication of data to an external database can enable report printing or further assessment of the patient's visual test data. For example, data collected and corresponding test results may be wirelessly transmitted and stored in a remote database accessible by authorized medical professionals.

It should be understood that, while FIG. 13 depicts the system 1300 as including a single vision screening system, in additional examples, the system 1300 may include any number of local or remote vision screening systems substantially similar to the vision screening system 1310 and configured to operate independently and/or in combination and configured to communicate via the network 1308.

As discussed herein, FIG. 13 depicts a vision screening device 1304 that includes components for administering vision screening tests to a patient. In some examples, one or more components may be implemented on a remote vision screening system communicating with the vision screening device 1304 over a network 1308.

The foregoing is merely illustrative of the principles of this disclosure and various modifications can be made by those skilled in the art without departing from the scope of this disclosure. The above-described examples are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

As a further example, variations of apparatus or process limitations (e.g., dimensions, configurations, components, process step order, etc.) can be made to further optimize the provided structures, devices and methods, as shown and described herein. In any event, the structures and devices, as well as the associated methods, described herein have many applications. Therefore, the disclosed subject matter should not be limited to any single example described herein, but rather should be construed in breadth and scope in accordance with the appended claims.

### EXAMPLE CLAUSES

The following paragraphs are provided to illustrate various aspects of the present disclosure:
A: An assembly, comprising: a housing having an emission surface; a first emitter disposed within the housing and including one or more light sources controllable by a processing device of the vision screening device; a reflector connected to the first emitter and partially surrounding the one or more light sources, the reflector configured to reflect from the one or more light sources out of the emission surface; a polarizer coupled to the reflector at an output side of the reflector; a beam splitter positioned between the polarizer and the emission surface such that light from the reflector passes through the beam splitter and reflected light received through the emission surface is directed at an angle relative to an incident angle of the reflected light; an imaging system of the assembly configured to receive the reflected light from the beam splitter; and one or more computing systems configured to perform a vision screening evaluation based on image data received from the imaging system.
B: The assembly of paragraph A, further comprising a light trap disposed within the housing and configured to prevent stray light from reflecting into the imaging system.
C: The assembly of any of paragraphs A-B, wherein the reflector defines one or more recesses that at least partially surround the one or more light sources to reflect light emitted by the one or more light sources into columnar light towards the emission surface.
D: The assembly of any of paragraphs A-C, wherein the one or more recesses comprise a parabolic reflector.
E: The assembly of any of paragraphs A-D, wherein the one or more recesses further comprise a beveled undercut adjacent a connection between the reflector and the first emitter.
F: The assembly of any of paragraphs A-E, further comprising a second emitter disposed between the beam splitter and the emission surface, including one or more second light sources, and defining a hole positioned to: enable emitted light from the first emitter to pass through and exit through the emission surface; and enable the reflected light to pass through to the beam splitter.
G: The assembly of any of paragraphs A-F, wherein the reflector further comprises three mounting locations configured to secure the polarizer to the reflector, the three mounting locations disposed on the reflector at an unequal spacing to enable mounting of the polarizer in a single orientation.
H: A system comprising: an emitter having: a support; one or more LEDs arranged in an array on the support; and a reflector having an output side and a connection side where the reflector is mounted to the support, the reflector partially surrounding the one or more LEDs and configured to reflect emitted light from the one or more LEDs in a columnar direction; a polarizer coupled to the reflector at the output side; a beam splitter positioned downstream of the polarizer such that light from the reflector passes through the beam splitter along an incident axis and reflected light received at the beam splitter is directed at an angle relative to the incident axis; and an imaging system configured to receive the reflected light from the beam splitter and produce image data.
I: The system of paragraph H, further comprising a light trap disposed within a housing surrounding the system and configured to receive reflected light and prevent light from reflecting from a source other than the beam splitter into the imaging system.
J: The system of any of paragraphs H-I, wherein the reflector defines one or more recesses arranged in the array that at least partially surround the one or more LEDs to reflect light emitted by the one or more LEDs.
K: The system of any of paragraphs H-J, wherein the one or more recesses comprise a parabolic reflector.
L: The system of any of paragraphs H-K, wherein the reflector is formed of a metal and is thermally connected to the support and is configured to act as a heat sink for the one or more LEDs and one or more electronic components of the emitter.
M: The system of any of paragraphs H-L, wherein the emitter board is a first emitter board further comprising a second emitter disposed downstream of the beam splitter, including one or more second light sources, and defining a hole positioned to: enable emitted light from the first emitter to pass through; and enable the reflected light to pass through to the beam splitter.
N: The system of any of paragraphs H-M, wherein the reflector further comprises three mounting locations configured to secure the polarizer to the reflector, the three mounting locations disposed on the reflector at an unequal spacing to enable mounting of the polarizer in a single orientation.
O: A light system comprising: an emitter having: a support; one or more LEDs arranged in an array on the support; and a reflector having an output side and a connection side where the reflector is mounted to the support, the reflector partially surrounding the one or more LEDs and configured to reflect emitted light from the one or more LEDs in a columnar direction; a polarizer coupled to the reflector at the output side; a beam splitter positioned downstream of the polarizer such that light from the reflector passes through the beam splitter along an incident axis and reflected light received at the beam splitter is directed at an angle relative to the incident axis; and an imaging system configured to receive the reflected light from the beam splitter and produce image data.
P: The system of paragraph O, wherein the reflector further comprises three mounting locations arranged at an unequal spacing about a perimeter of a mounting circle, the three mounting locations disposed to enable mounting of the polarizer in a single orientation.
Q: The system of any of paragraphs O-P, wherein the emitter is a first emitter and further comprising a second emitter disposed downstream of the beam splitter, including one or more second light sources, and defining a hole positioned to: enable emitted light from the first emitter to pass through; and enable the reflected light to pass through to the beam splitter.
R: The light emitting system of any of paragraphs O-Q, wherein the reflector defines one or more recesses that at least partially surround the one or more LEDs to reflect light emitted by the one or more LEDs into columnar light.
S: The light emitting system of any of paragraphs O-R, wherein the one or more recesses comprise a parabolic reflector.
T: The light emitting system of any of paragraphs O-S, wherein the one or more recesses further comprise a beveled undercut adjacent a connection between the reflector and the emitter board.

What is claimed is:

1. An assembly, comprising:
a housing having an emission surface;
a first emitter disposed within the housing and including one or more light sources controllable by a processing device of the vision screening device;
a reflector connected to the first emitter and partially surrounding the one or more light sources, the reflector configured to reflect from the one or more light sources out of the emission surface;
a polarizer coupled to the reflector at an output side of the reflector;
a beam splitter positioned between the polarizer and the emission surface such that light from the reflector passes through the beam splitter and reflected light received through the emission surface is directed at an angle relative to an incident angle of the reflected light;
an imaging system of the assembly configured to receive the reflected light from the beam splitter; and
one or more computing systems configured to perform a vision screening evaluation based on image data received from the imaging system.

2. The assembly of claim 1, further comprising a light trap disposed within the housing and configured to prevent stray light from reflecting into the imaging system.

3. The assembly of claim 1, wherein the reflector defines one or more recesses that at least partially surround the one or more light sources to reflect light emitted by the one or more light sources into columnar light towards the emission surface.

4. The assembly of claim 3, wherein the one or more recesses comprise a parabolic reflector.

5. The assembly of claim 3, wherein the one or more recesses further comprise a beveled undercut adjacent a connection between the reflector and the first emitter.

6. The assembly of claim 1, further comprising a second emitter disposed between the beam splitter and the emission surface, including one or more second light sources, and defining a hole positioned to:
enable emitted light from the first emitter to pass through and exit through the emission surface; and
enable the reflected light to pass through to the beam splitter.

7. The assembly of claim 1, wherein the reflector further comprises three mounting locations configured to secure the polarizer to the reflector, the three mounting locations disposed on the reflector at an unequal spacing to enable mounting of the polarizer in a single orientation.

8. A system comprising:
an emitter having:
a support;
one or more LEDs arranged in an array on the support; and
a reflector having an output side and a connection side where the reflector is mounted to the support, the reflector partially surrounding the one or more LEDs and configured to reflect emitted light from the one or more LEDs in a columnar direction;
a polarizer coupled to the reflector at the output side;
a beam splitter positioned downstream of the polarizer such that light from the reflector passes through the beam splitter along an incident axis and reflected light received at the beam splitter is directed at an angle relative to the incident axis; and
an imaging system configured to receive the reflected light from the beam splitter and produce image data.

9. The system of claim 8, further comprising a light trap disposed within a housing surrounding the system and configured to receive reflected light and prevent light from reflecting from a source other than the beam splitter into the imaging system.

10. The system of claim 8, wherein the reflector defines one or more recesses arranged in the array that at least partially surround the one or more LEDs to reflect light emitted by the one or more LEDs.

11. The system of claim 10, wherein the one or more recesses comprise a parabolic reflector.

12. The system of claim 8, wherein the reflector is formed of a metal and is thermally connected to the support and is configured to act as a heat sink for the one or more LEDs and one or more electronic components of the emitter.

13. The system of claim 8, wherein the emitter board is a first emitter board further comprising a second emitter disposed downstream of the beam splitter, including one or more second light sources, and defining a hole positioned to:
enable emitted light from the first emitter to pass through; and
enable the reflected light to pass through to the beam splitter.

14. The system of claim 8, wherein the reflector further comprises three mounting locations configured to secure the polarizer to the reflector, the three mounting locations disposed on the reflector at an unequal spacing to enable mounting of the polarizer in a single orientation.

15. A light system comprising:
an emitter having:
a support;
one or more LEDs arranged in an array on the support; and
a reflector having an output side and a connection side where the reflector is mounted to the support, the reflector partially surrounding the one or more LEDs and configured to reflect emitted light from the one or more LEDs in a columnar direction;
a polarizer coupled to the reflector at the output side;
a beam splitter positioned downstream of the polarizer such that light from the reflector passes through the beam splitter along an incident axis and reflected light received at the beam splitter is directed at an angle relative to the incident axis; and
an imaging system configured to receive the reflected light from the beam splitter and produce image data.

16. The system of claim 15, wherein the reflector further comprises three mounting locations arranged at an unequal spacing about a perimeter of a mounting circle, the three mounting locations disposed to enable mounting of the polarizer in a single orientation.

17. The system of claim 15, wherein the emitter is a first emitter and further comprising a second emitter disposed downstream of the beam splitter, including one or more second light sources, and defining a hole positioned to:
enable emitted light from the first emitter to pass through; and
enable the reflected light to pass through to the beam splitter.

18. The light emitting system of claim 15, wherein the reflector defines one or more recesses that at least partially surround the one or more LEDs to reflect light emitted by the one or more LEDs into columnar light.

19. The light emitting system of claim 18, wherein the one or more recesses comprise a parabolic reflector.

20. The light emitting system of claim 18, wherein the one or more recesses further comprise a beveled undercut adjacent a connection between the reflector and the emitter board.
